SEQUENCE LISTING (34 sequences)

SEQ ID NO: 1 - DNA, 2412 bp, Artificial Sequence
  Synthetic sequence, EFMvaE
atgaaaaccgtggttattatcgatgcgctgcgcacgccgattggtaaatataaaggcagcctgtctcaagtgagcgccgttgatctgggtacgcatgtgaccacgcagctgctgaaacgtcacagcaccatctctgaagaaattgatcaggtgatctttggtaacgttctgcaagccggtaatggtcagaatccggcacgtcagattgcaatcaacagtggcctgagccatgaaattccggcgatgaccgtgaatgaagtttgcggtagcggcatgaaagcggttattctggccaaacagctgatccagctgggtgaagcggaagtgctgattgccggcggtatcgaaaacatgagtcaggcaccgaaactgcaacgttttaattatgaaaccgaaagctacgatgccccgttcagctctatgatgtatgatggcctgaccgatgcatttagcggtcaggcgatgggcctgacggcagaaaacgtggcggaaaaataccatgttacccgcgaagaacaggatcagttttctgttcacagtcagctgaaagcggcccaggcccaggcagaaggtattttcgccgatgaaatcgcaccgctggaagtgtctggtacgctggttgaaaaagatgaaggcattcgtccgaatagtagcgtggaaaaactgggcaccctgaaaacggtgttcaaagaagatggcaccgttacggcgggcaatgcaagcaccatcaatgatggtgcgagtgccctgattatcgcgagccaggaatatgcagaagcgcatggcctgccgtacctggccattatccgcgattctgtggaagttggtattgatccggcatatatgggcattagtccgatcaaagcgattcagaaactgctggcccgtaaccagctgaccaccgaagaaattgatctgtacgaaatcaatgaagcgtttgcagcgaccagtattgtggttcagcgcgaactggccctgccggaagaaaaagttaacatttatggcggtggcatcagcctgggtcacgcaattggtgccaccggtgcacgtctgctgaccagtctgagctatcagctgaatcagaaagagaaaaaatacggtgtggcaagcctgtgtattggtggcggtctgggtctggccatgctgctggaacgtccgcagcagaagaaaaactctcgtttttaccagatgagtccggaagaacgtctggccagtctgctgaacgaaggccagattagcgcagataccaaaaaagaattcgaaaatacggcactgtctagtcagatcgcgaaccatatgattgaaaatcagatcagcgaaaccgaagtgccgatgggtgttggcctgcacctgaccgtggatgaaacggattatctggttccgatggcgacggaagaaccgagcgttattgccgcactgtctaacggtgcaaaaatcgcgcagggctttaaaaccgtgaatcagcagcgtctgatgcgcggccagattgtgttctacgatgttgcggatccggaaagcctgatcgataaactgcaagtgcgcgaagccgaagtttttcagcaggcagaactgagctatccgtctattgtgaaacgtggcggtggcctgcgcgatctgcaataccgtacctttgatgaaagtttcgtgagcgttgatttcctggtggatgttaaagatgccatgggtgcaaacatcgtgaatgcgatgctggaaggcgttgccgaactgtttcgtgaatggttcgcggaacagaaaatcctgttttctatcctgagtaactacgcgaccgaaagcgtggttaccatgaaaacggccattcctgtgagccgcctgtctaaaggtagtaatggccgtgaaattgcggaaaaaatcgttctggcgagccgctatgcctctctggatccgtaccgtgccgtgacccataacaaaggtattatgaatggcatcgaagcagtggttctggcgaccggtaacgatacccgtgccgtgtctgcaagttgccatgcattcgcagttaaagaaggtcgttatcagggcctgaccagctggacgctggatggtgaacagctgatcggcgaaatttctgtgccgctggccctggcaaccgtgggtggcgcgacgaaagttctgccgaaaagccaggcggccgcagatctgctggcggtgaccgatgcaaaagaactgtctcgcgtggttgcggccgttggtctggcacagaatctggcagcgctgcgtgcgctggtgtctgaaggtattcagaaaggccacatggcactgcaagcccgtagtctggccatgaccgtgggtgcaacgggcaaagaagtggaagcagttgcgcagcagctgaaacgccagaaaaccatgaaccaggatcgtgccatggcaatcctgaatgatctgcgcaaacagtaa

SEQ ID NO: 2 - DNA, 1152 bp, Artificial Sequence
  Synthetic sequence, EFMvaS
atgaccattggtatcgataaaattagctttttcgtgccgccgtattacatcgatatgacggcgctggccgaagcacgtaacgttgatccgggcaaatttcatattggcatcggtcaggatcagatggcggtgaacccgatttctcaggatatcgttaccttcgcggccaatgcagcggaagcaattctgacgaaagaagataaagaagcgattgatatggtgatcgttggcaccgaaagctctatcgatgaaagtaaagccgcagcggtggttctgcaccgtctgatgggcattcagccgtttgcgcgcagcttcgaaatcaaagaagcctgctatggcgcgaccgccggtctgcaactggccaaaaaccatgtggcactgcacccggataaaaaagttctggtggttgccgcagatattgcgaaatacggtctgaatagcggcggtgaaccgacccagggtgcaggtgccgtggcaatgctggttgcatctgaaccgcgtattctggcgctgaaagaagataacgtgatgctgacccaggatatctatgatttttggcgtccgaccggtcatccgtacccgatggtggatggcccgctgagtaatgaaacctatattcagagcttcgcccaggtttgggatgaacataaaaaacgtacgggtctggattttgcggattatgatgcactggcgttccacattccgtacaccaaaatgggcaaaaaagcgctgctggccaaaatcagcgatcagacggaagccgaacaggaacgtattctggcacgctatgaagaaagcatcgtgtactctcgtcgcgttggcaacctgtataccggttctctgtacctgggcctgattagtctgctggaaaacgcgaccacgctgacggccggcaatcagatcggtctgttttcttatggcagtggtgccgtggcagaatttttcaccggtgaactggttgccggctaccagaaccatctgcaaaaagaaacccacctggccctgctggataatcgcacggaactgtctattgcagaatatgaagcaatgtttgcggaaaccctggatacggatatcgatcagaccctggaagatgaactgaaatatagtattagcgcgatcaacaatacggtgcgtagttaccgcaattaa

SEQ ID NO: 3 - DNA, 5314 bp, Artificial Sequence
  Synthetic sequence, pUC57-EFmvaE
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcaggaggtaaaaaaacatgaaaaccgtggttattatcgatgcgctgcgcacgccgattggtaaatataaaggcagcctgtctcaagtgagcgccgttgatctgggtacgcatgtgaccacgcagctgctgaaacgtcacagcaccatctctgaagaaattgatcaggtgatctttggtaacgttctgcaagccggtaatggtcagaatccggcacgtcagattgcaatcaacagtggcctgagccatgaaattccggcgatgaccgtgaatgaagtttgcggtagcggcatgaaagcggttattctggccaaacagctgatccagctgggtgaagcggaagtgctgattgccggcggtatcgaaaacatgagtcaggcaccgaaactgcaacgttttaattatgaaaccgaaagctacgatgccccgttcagctctatgatgtatgatggcctgaccgatgcatttagcggtcaggcgatgggcctgacggcagaaaacgtggcggaaaaataccatgttacccgcgaagaacaggatcagttttctgttcacagtcagctgaaagcggcccaggcccaggcagaaggtattttcgccgatgaaatcgcaccgctggaagtgtctggtacgctggttgaaaaagatgaaggcattcgtccgaatagtagcgtggaaaaactgggcaccctgaaaacggtgttcaaagaagatggcaccgttacggcgggcaatgcaagcaccatcaatgatggtgcgagtgccctgattatcgcgagccaggaatatgcagaagcgcatggcctgccgtacctggccattatccgcgattctgtggaagttggtattgatccggcatatatgggcattagtccgatcaaagcgattcagaaactgctggcccgtaaccagctgaccaccgaagaaattgatctgtacgaaatcaatgaagcgtttgcagcgaccagtattgtggttcagcgcgaactggccctgccggaagaaaaagttaacatttatggcggtggcatcagcctgggtcacgcaattggtgccaccggtgcacgtctgctgaccagtctgagctatcagctgaatcagaaagagaaaaaatacggtgtggcaagcctgtgtattggtggcggtctgggtctggccatgctgctggaacgtccgcagcagaagaaaaactctcgtttttaccagatgagtccggaagaacgtctggccagtctgctgaacgaaggccagattagcgcagataccaaaaaagaattcgaaaatacggcactgtctagtcagatcgcgaaccatatgattgaaaatcagatcagcgaaaccgaagtgccgatgggtgttggcctgcacctgaccgtggatgaaacggattatctggttccgatggcgacggaagaaccgagcgttattgccgcactgtctaacggtgcaaaaatcgcgcagggctttaaaaccgtgaatcagcagcgtctgatgcgcggccagattgtgttctacgatgttgcggatccggaaagcctgatcgataaactgcaagtgcgcgaagccgaagtttttcagcaggcagaactgagctatccgtctattgtgaaacgtggcggtggcctgcgcgatctgcaataccgtacctttgatgaaagtttcgtgagcgttgatttcctggtggatgttaaagatgccatgggtgcaaacatcgtgaatgcgatgctggaaggcgttgccgaactgtttcgtgaatggttcgcggaacagaaaatcctgttttctatcctgagtaactacgcgaccgaaagcgtggttaccatgaaaacggccattcctgtgagccgcctgtctaaaggtagtaatggccgtgaaattgcggaaaaaatcgttctggcgagccgctatgcctctctggatccgtaccgtgccgtgacccataacaaaggtattatgaatggcatcgaagcagtggttctggcgaccggtaacgatacccgtgccgtgtctgcaagttgccatgcattcgcagttaaagaaggtcgttatcagggcctgaccagctggacgctggatggtgaacagctgatcggcgaaatttctgtgccgctggccctggcaaccgtgggtggcgcgacgaaagttctgccgaaaagccaggcggccgcagatctgctggcggtgaccgatgcaaaagaactgtctcgcgtggttgcggccgttggtctggcacagaatctggcagcgctgcgtgcgctggtgtctgaaggtattcagaaaggccacatggcactgcaagcccgtagtctggccatgaccgtgggtgcaacgggcaaagaagtggaagcagttgcgcagcagctgaaacgccagaaaaccatgaaccaggatcgtgccatggcaatcctgaatgatctgcgcaaacagtaaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - DNA, 4054 bp, Artificial Sequence
  Synthetic sequence, pUC57-EFmvaS
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcaggaggtaaaaaaacatgaccattggtatcgataaaattagctttttcgtgccgccgtattacatcgatatgacggcgctggccgaagcacgtaacgttgatccgggcaaatttcatattggcatcggtcaggatcagatggcggtgaacccgatttctcaggatatcgttaccttcgcggccaatgcagcggaagcaattctgacgaaagaagataaagaagcgattgatatggtgatcgttggcaccgaaagctctatcgatgaaagtaaagccgcagcggtggttctgcaccgtctgatgggcattcagccgtttgcgcgcagcttcgaaatcaaagaagcctgctatggcgcgaccgccggtctgcaactggccaaaaaccatgtggcactgcacccggataaaaaagttctggtggttgccgcagatattgcgaaatacggtctgaatagcggcggtgaaccgacccagggtgcaggtgccgtggcaatgctggttgcatctgaaccgcgtattctggcgctgaaagaagataacgtgatgctgacccaggatatctatgatttttggcgtccgaccggtcatccgtacccgatggtggatggcccgctgagtaatgaaacctatattcagagcttcgcccaggtttgggatgaacataaaaaacgtacgggtctggattttgcggattatgatgcactggcgttccacattccgtacaccaaaatgggcaaaaaagcgctgctggccaaaatcagcgatcagacggaagccgaacaggaacgtattctggcacgctatgaagaaagcatcgtgtactctcgtcgcgttggcaacctgtataccggttctctgtacctgggcctgattagtctgctggaaaacgcgaccacgctgacggccggcaatcagatcggtctgttttcttatggcagtggtgccgtggcagaatttttcaccggtgaactggttgccggctaccagaaccatctgcaaaaagaaacccacctggccctgctggataatcgcacggaactgtctattgcagaatatgaagcaatgtttgcggaaaccctggatacggatatcgatcagaccctggaagatgaactgaaatatagtattagcgcgatcaacaatacggtgcgtagttaccgcaattaaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - DNA, 38 bp, Artificial Sequence
  Synthetic sequence, primer to amplify trc promoter (Ptrc)
gaattcgagctcggtacccttgacaattaatcatccgg

SEQ ID NO: 6 - DNA, 45 bp, Artificial Sequence
  Synthetic sequence, primer to amplify trc promoter (Ptrc)
aaccacggttttcatgtttttttacctcctgagctcggatccatg

SEQ ID NO: 7 - DNA, 45 bp, Artificial Sequence
  Synthetic sequence, primer to amplify mvaE
catggatccgagctcaggaggtaaaaaaacatgaaaaccgtggtt

SEQ ID NO: 8 - DNA, 55 bp, Artificial Sequence
  Synthetic sequence, primer to amplify mvaE, mvaS
ttatcgataccaatggtcatgtttttttacctcctttactgtttgcgcagatcat

SEQ ID NO: 9 - DNA, 55 bp, Artificial Sequence
  Synthetic sequence, primer to amplify mvaS
atgatctgcgcaaacagtaaaggaggtaaaaaaacatgaccattggtatcgataa

SEQ ID NO: 10 - DNA, 40 bp, Artificial Sequence
  Synthetic sequence, primer to amplify tryptophane terminator (Ttrp)
cagcggaactggcggctcccttaattgcggtaactacgca

SEQ ID NO: 11 - DNA, 40 bp, Artificial Sequence
  Synthetic sequence, primer to amplify tryptophane terminator (Ttrp)
tgcgtagttaccgcaattaagggagccgccagttccgctg

SEQ ID NO: 12 - DNA, 38 bp, Artificial Sequence
  Synthetic sequence, primer
gtcgactctagaggatccctaatgagaattagtcaaat

SEQ ID NO: 13 - DNA, 8053 bp, Artificial Sequence
  Synthetic sequence, pMW-Ptrc-mvaES-Ttrp
gacagtaagacgggtaagcctgttgatgataccgctgccttactgggtgcattagccagtctgaatgacctgtcacgggataatccgaagtggtcagactggaaaatcagagggcaggaactgcagaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcaggaggtaaaaaaacatgaaaaccgtggttattatcgatgcgctgcgcacgccgattggtaaatataaaggcagcctgtctcaagtgagcgccgttgatctgggtacgcatgtgaccacgcagctgctgaaacgtcacagcaccatctctgaagaaattgatcaggtgatctttggtaacgttctgcaagccggtaatggtcagaatccggcacgtcagattgcaatcaacagtggcctgagccatgaaattccggcgatgaccgtgaatgaagtttgcggtagcggcatgaaagcggttattctggccaaacagctgatccagctgggtgaagcggaagtgctgattgccggcggtatcgaaaacatgagtcaggcaccgaaactgcaacgttttaattatgaaaccgaaagctacgatgccccgttcagctctatgatgtatgatggcctgaccgatgcatttagcggtcaggcgatgggcctgacggcagaaaacgtggcggaaaaataccatgttacccgcgaagaacaggatcagttttctgttcacagtcagctgaaagcggcccaggcccaggcagaaggtattttcgccgatgaaatcgcaccgctggaagtgtctggtacgctggttgaaaaagatgaaggcattcgtccgaatagtagcgtggaaaaactgggcaccctgaaaacggtgttcaaagaagatggcaccgttacggcgggcaatgcaagcaccatcaatgatggtgcgagtgccctgattatcgcgagccaggaatatgcagaagcgcatggcctgccgtacctggccattatccgcgattctgtggaagttggtattgatccggcatatatgggcattagtccgatcaaagcgattcagaaactgctggcccgtaaccagctgaccaccgaagaaattgatctgtacgaaatcaatgaagcgtttgcagcgaccagtattgtggttcagcgcgaactggccctgccggaagaaaaagttaacatttatggcggtggcatcagcctgggtcacgcaattggtgccaccggtgcacgtctgctgaccagtctgagctatcagctgaatcagaaagagaaaaaatacggtgtggcaagcctgtgtattggtggcggtctgggtctggccatgctgctggaacgtccgcagcagaagaaaaactctcgtttttaccagatgagtccggaagaacgtctggccagtctgctgaacgaaggccagattagcgcagataccaaaaaagaattcgaaaatacggcactgtctagtcagatcgcgaaccatatgattgaaaatcagatcagcgaaaccgaagtgccgatgggtgttggcctgcacctgaccgtggatgaaacggattatctggttccgatggcgacggaagaaccgagcgttattgccgcactgtctaacggtgcaaaaatcgcgcagggctttaaaaccgtgaatcagcagcgtctgatgcgcggccagattgtgttctacgatgttgcggatccggaaagcctgatcgataaactgcaagtgcgcgaagccgaagtttttcagcaggcagaactgagctatccgtctattgtgaaacgtggcggtggcctgcgcgatctgcaataccgtacctttgatgaaagtttcgtgagcgttgatttcctggtggatgttaaagatgccatgggtgcaaacatcgtgaatgcgatgctggaaggcgttgccgaactgtttcgtgaatggttcgcggaacagaaaatcctgttttctatcctgagtaactacgcgaccgaaagcgtggttaccatgaaaacggccattcctgtgagccgcctgtctaaaggtagtaatggccgtgaaattgcggaaaaaatcgttctggcgagccgctatgcctctctggatccgtaccgtgccgtgacccataacaaaggtattatgaatggcatcgaagcagtggttctggcgaccggtaacgatacccgtgccgtgtctgcaagttgccatgcattcgcagttaaagaaggtcgttatcagggcctgaccagctggacgctggatggtgaacagctgatcggcgaaatttctgtgccgctggccctggcaaccgtgggtggcgcgacgaaagttctgccgaaaagccaggcggccgcagatctgctggcggtgaccgatgcaaaagaactgtctcgcgtggttgcggccgttggtctggcacagaatctggcagcgctgcgtgcgctggtgtctgaaggtattcagaaaggccacatggcactgcaagcccgtagtctggccatgaccgtgggtgcaacgggcaaagaagtggaagcagttgcgcagcagctgaaacgccagaaaaccatgaaccaggatcgtgccatggcaatcctgaatgatctgcgcaaacagtaaaggaggtaaaaaaacatgaccattggtatcgataaaattagctttttcgtgccgccgtattacatcgatatgacggcgctggccgaagcacgtaacgttgatccgggcaaatttcatattggcatcggtcaggatcagatggcggtgaacccgatttctcaggatatcgttaccttcgcggccaatgcagcggaagcaattctgacgaaagaagataaagaagcgattgatatggtgatcgttggcaccgaaagctctatcgatgaaagtaaagccgcagcggtggttctgcaccgtctgatgggcattcagccgtttgcgcgcagcttcgaaatcaaagaagcctgctatggcgcgaccgccggtctgcaactggccaaaaaccatgtggcactgcacccggataaaaaagttctggtggttgccgcagatattgcgaaatacggtctgaatagcggcggtgaaccgacccagggtgcaggtgccgtggcaatgctggttgcatctgaaccgcgtattctggcgctgaaagaagataacgtgatgctgacccaggatatctatgatttttggcgtccgaccggtcatccgtacccgatggtggatggcccgctgagtaatgaaacctatattcagagcttcgcccaggtttgggatgaacataaaaaacgtacgggtctggattttgcggattatgatgcactggcgttccacattccgtacaccaaaatgggcaaaaaagcgctgctggccaaaatcagcgatcagacggaagccgaacaggaacgtattctggcacgctatgaagaaagcatcgtgtactctcgtcgcgttggcaacctgtataccggttctctgtacctgggcctgattagtctgctggaaaacgcgaccacgctgacggccggcaatcagatcggtctgttttcttatggcagtggtgccgtggcagaatttttcaccggtgaactggttgccggctaccagaaccatctgcaaaaagaaacccacctggccctgctggataatcgcacggaactgtctattgcagaatatgaagcaatgtttgcggaaaccctggatacggatatcgatcagaccctggaagatgaactgaaatatagtattagcgcgatcaacaatacggtgcgtagttaccgcaattaagggagccgccagttccgctggcggcattttaactttctttaatgaagccggaaaaatcctaaattcatttaatatttatctttttaccgtttcgcttaccccggtcgaacgtcaacttacgtcatttttccgcccaacagtaatataatcaaacaaattaatcccgcaacataacaccagtaaaatcaataattttctctaagtcacttattcctcaggtaattgttaatatatccagaatgttcctcaaaatatattttccctctatcttctcgttgcgcttaatttgactaattctcattagggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctttctcatagctcacgctgtaggtatctcagttcggtgtaggtcgttcgctccaagctgggctgtgtgccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgccaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcactaccgcattaaagcatatcgatgataagctgtcaaacatgagcgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 14 - DNA, 40 bp, Artificial Sequence
  Synthetic sequence, primer to amplify pMW-Ptrc-mvaES-Ttrp
ctctaaggaggttataaaaaatgaaaaccgtggttattat

SEQ ID NO: 15 - DNA, 40 bp, Artificial Sequence
  Synthetic sequence, primer to amplify pMW-Ptrc-mvaES-Ttrp
aatattgaaaaaggaagagtgggtaccgagctcgaattca

SEQ ID NO: 16 - DNA, 40 bp, Artificial Sequence
  Synthetic sequence, primer to amplify pKD46
tgaattcgagctcggtacccactcttcctttttcaatatt

SEQ ID NO: 17 - DNA, 40 bp, Artificial Sequence
  Synthetic sequence, primer to amplify pKD46
ataataaccacggttttcattttttataacctccttagag

SEQ ID NO: 18 - DNA, 9180 bp, Artificial Sequence
  Synthetic sequence, pMW-Para-mvaES-Ttrp
gacagtaagacgggtaagcctgttgatgataccgctgccttactgggtgcattagccagtctgaatgacctgtcacgggataatccgaagtggtcagactggaaaatcagagggcaggaactgcagaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccactcttcctttttcaatattattgaagcatttatcagggttattgtctcatgagcggatacatatttgaatgtatttagaaaaataaacaaataggggttccgcgcacatttccccgaaaagtgccacctgcatcgatttattatgacaacttgacggctacatcattcactttttcttcacaaccggcacggaactcgctcgggctggccccggtgcattttttaaatacccgcgagaagtagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctctaaggaggttataaaaaatgaaaaccgtggttattatcgatgcgctgcgcacgccgattggtaaatataaaggcagcctgtctcaagtgagcgccgttgatctgggtacgcatgtgaccacgcagctgctgaaacgtcacagcaccatctctgaagaaattgatcaggtgatctttggtaacgttctgcaagccggtaatggtcagaatccggcacgtcagattgcaatcaacagtggcctgagccatgaaattccggcgatgaccgtgaatgaagtttgcggtagcggcatgaaagcggttattctggccaaacagctgatccagctgggtgaagcggaagtgctgattgccggcggtatcgaaaacatgagtcaggcaccgaaactgcaacgttttaattatgaaaccgaaagctacgatgccccgttcagctctatgatgtatgatggcctgaccgatgcatttagcggtcaggcgatgggcctgacggcagaaaacgtggcggaaaaataccatgttacccgcgaagaacaggatcagttttctgttcacagtcagctgaaagcggcccaggcccaggcagaaggtattttcgccgatgaaatcgcaccgctggaagtgtctggtacgctggttgaaaaagatgaaggcattcgtccgaatagtagcgtggaaaaactgggcaccctgaaaacggtgttcaaagaagatggcaccgttacggcgggcaatgcaagcaccatcaatgatggtgcgagtgccctgattatcgcgagccaggaatatgcagaagcgcatggcctgccgtacctggccattatccgcgattctgtggaagttggtattgatccggcatatatgggcattagtccgatcaaagcgattcagaaactgctggcccgtaaccagctgaccaccgaagaaattgatctgtacgaaatcaatgaagcgtttgcagcgaccagtattgtggttcagcgcgaactggccctgccggaagaaaaagttaacatttatggcggtggcatcagcctgggtcacgcaattggtgccaccggtgcacgtctgctgaccagtctgagctatcagctgaatcagaaagagaaaaaatacggtgtggcaagcctgtgtattggtggcggtctgggtctggccatgctgctggaacgtccgcagcagaagaaaaactctcgtttttaccagatgagtccggaagaacgtctggccagtctgctgaacgaaggccagattagcgcagataccaaaaaagaattcgaaaatacggcactgtctagtcagatcgcgaaccatatgattgaaaatcagatcagcgaaaccgaagtgccgatgggtgttggcctgcacctgaccgtggatgaaacggattatctggttccgatggcgacggaagaaccgagcgttattgccgcactgtctaacggtgcaaaaatcgcgcagggctttaaaaccgtgaatcagcagcgtctgatgcgcggccagattgtgttctacgatgttgcggatccggaaagcctgatcgataaactgcaagtgcgcgaagccgaagtttttcagcaggcagaactgagctatccgtctattgtgaaacgtggcggtggcctgcgcgatctgcaataccgtacctttgatgaaagtttcgtgagcgttgatttcctggtggatgttaaagatgccatgggtgcaaacatcgtgaatgcgatgctggaaggcgttgccgaactgtttcgtgaatggttcgcggaacagaaaatcctgttttctatcctgagtaactacgcgaccgaaagcgtggttaccatgaaaacggccattcctgtgagccgcctgtctaaaggtagtaatggccgtgaaattgcggaaaaaatcgttctggcgagccgctatgcctctctggatccgtaccgtgccgtgacccataacaaaggtattatgaatggcatcgaagcagtggttctggcgaccggtaacgatacccgtgccgtgtctgcaagttgccatgcattcgcagttaaagaaggtcgttatcagggcctgaccagctggacgctggatggtgaacagctgatcggcgaaatttctgtgccgctggccctggcaaccgtgggtggcgcgacgaaagttctgccgaaaagccaggcggccgcagatctgctggcggtgaccgatgcaaaagaactgtctcgcgtggttgcggccgttggtctggcacagaatctggcagcgctgcgtgcgctggtgtctgaaggtattcagaaaggccacatggcactgcaagcccgtagtctggccatgaccgtgggtgcaacgggcaaagaagtggaagcagttgcgcagcagctgaaacgccagaaaaccatgaaccaggatcgtgccatggcaatcctgaatgatctgcgcaaacagtaaaggaggtaaaaaaacatgaccattggtatcgataaaattagctttttcgtgccgccgtattacatcgatatgacggcgctggccgaagcacgtaacgttgatccgggcaaatttcatattggcatcggtcaggatcagatggcggtgaacccgatttctcaggatatcgttaccttcgcggccaatgcagcggaagcaattctgacgaaagaagataaagaagcgattgatatggtgatcgttggcaccgaaagctctatcgatgaaagtaaagccgcagcggtggttctgcaccgtctgatgggcattcagccgtttgcgcgcagcttcgaaatcaaagaagcctgctatggcgcgaccgccggtctgcaactggccaaaaaccatgtggcactgcacccggataaaaaagttctggtggttgccgcagatattgcgaaatacggtctgaatagcggcggtgaaccgacccagggtgcaggtgccgtggcaatgctggttgcatctgaaccgcgtattctggcgctgaaagaagataacgtgatgctgacccaggatatctatgatttttggcgtccgaccggtcatccgtacccgatggtggatggcccgctgagtaatgaaacctatattcagagcttcgcccaggtttgggatgaacataaaaaacgtacgggtctggattttgcggattatgatgcactggcgttccacattccgtacaccaaaatgggcaaaaaagcgctgctggccaaaatcagcgatcagacggaagccgaacaggaacgtattctggcacgctatgaagaaagcatcgtgtactctcgtcgcgttggcaacctgtataccggttctctgtacctgggcctgattagtctgctggaaaacgcgaccacgctgacggccggcaatcagatcggtctgttttcttatggcagtggtgccgtggcagaatttttcaccggtgaactggttgccggctaccagaaccatctgcaaaaagaaacccacctggccctgctggataatcgcacggaactgtctattgcagaatatgaagcaatgtttgcggaaaccctggatacggatatcgatcagaccctggaagatgaactgaaatatagtattagcgcgatcaacaatacggtgcgtagttaccgcaattaagggagccgccagttccgctggcggcattttaactttctttaatgaagccggaaaaatcctaaattcatttaatatttatctttttaccgtttcgcttaccccggtcgaacgtcaacttacgtcatttttccgcccaacagtaatataatcaaacaaattaatcccgcaacataacaccagtaaaatcaataattttctctaagtcacttattcctcaggtaattgttaatatatccagaatgttcctcaaaatatattttccctctatcttctcgttgcgcttaatttgactaattctcattagggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctttctcatagctcacgctgtaggtatctcagttcggtgtaggtcgttcgctccaagctgggctgtgtgccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgccaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcactaccgcattaaagcatatcgatgataagctgtcaaacatgagcgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 19 - DNA, 33 bp, Artificial Sequence
  Synthetic sequence, n67
tgcgaagacgtcctcgtgaagaaggtgttgctg

SEQ ID NO: 20 - DNA, 36 bp, Artificial Sequence
  Synthetic sequence, n68
tgcgaagggccccgttgtgtctcaaaatctctgatg

SEQ ID NO: 21 - DNA, 3471 bp, Artificial Sequence
  Synthetic sequence, chemically synthesized DNA fragment retaining artificial KDyI operon with optimized codons
gcatgcaggaggtatgaatgtcagagttgcgtgccttcagtgccccagggaaagcgttactcgctggtggatatttagttttagatacaaaatatgaagcatttgtagtcggattatcggcacgtatgcacgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatcgtaacttgttcgttattgatattttctctgatgatgcctaccattctcaggaggatagcgttaccgaacatcgtggcaaccgccgtttgagttttcattcgcaccgtattgaagaagttcccaaaacagggctgggctcctcggcaggtttagtcacagttttaactacagctttggcctccttttttgtatcggacctggaaaataatgtagacaaatatcgtgaagttattcataatttagcacaagttgctcattgtcaagctcagggtaaaattggaagcgggtttgatgtagcggcggcagcatatggatctatccgttatcgccgtttcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcctcaaaatatatacagaactcgatcatgcaaattctcgttttatggatggactctctaaactcgatcgcttacacgagactcatgacgattacagcgatcagatatttgagtctcttgagcgtaatgactgtacctgtcaaaagtatcctgaaatcacagaagttcgtgatgcagttgccacaattcgtcgttcctttcgtaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttcgtgctcaaaccgctaatgacaaacgtttttctaaggttcaatggctggatgtaactcaggctgactggggtgttcgtaaagaaaaagatccggaaacttatcttgataaataactgcagaggaggtatgaatgaccgtttacacagcatccgttaccgcacccgtcaacatcgcaacccttaagtattgggggaaacgtgacacgaagttgaatctgcccaccaattcgtccatatcagtgactttatcgcaagatgacctccgtacgttgacctctgcggctactgcacctgagtttgaacgcgacactttgtggttaaatggagaaccacacagcatcgacaatgaacgtactcaaaattgtctgcgcgacctccgccaattacgtaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctcgtatagcacgtaaggggtctggttcagcttgtcgttcgttgtttggcggatacgtggcctgggaaatgggaaaagctgaagatggtcatgattccatggcagtacaaatcgcagacagctctgactggcctcagatgaaagcatgtgtccttgttgtcagcgatattaaaaaggatgtgagttccactcagggtatgcaattgaccgtggcaacctccgaactctttaaagaacgtattgaacatgtcgtaccaaagcgttttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgcccgtgtgattttaactcaagtcggttcaggcccacaagaaacaaacgaatctttgattgacgcaaagactggtctcccaaaggaataaggatccaggaggtatgaatgactgccgacaacaatagtatgccccatggtgcagtatctagttacgccaaattagtgcaaaaccaaacacctgaagacattttggaagagtttcctgaaattattccattacaacaacgtcctaatacccgctctagtgagacgtcaaatgacgaaagcggagaaacatgtttttctggtcatgatgaggagcaaattaagttaatgaatgaaaattgtattgttttggattgggacgataatgctattggtgccggcaccaagaaagtttgtcatttaatggaaaatattgaaaagggtttacttcatcgtgcattctccgtctttattttcaatgaacaaggtgaattacttttacaacaacgtgccactgaaaaaataactttccctgatctttggactaacacatgctgctctcatccactttgtattgatgacgaattaggtttgaagggtaagctcgacgataagattaagggcgctattactgcggcggtgcgtaaactcgatcatgaattaggtattccagaagatgaaactaagacacgtggtaagtttcactttttaaaccgtatccattacatggcaccaagcaatgaaccatggggtgaacatgaaattgattacatcctcttttataagatcaacgctaaagaaaacttgactgtcaacccaaacgtcaatgaagttcgtgacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgaccgtcaaattcatcgtatgctctaaggtacc

SEQ ID NO: 22 - DNA, 900 bp, Methanocella paludicola
atgacgatgtgttcagcccccggtaaggtctttttattcggcgagcatgcggtcgtttacggcaagcgcgcgatcgcatgcgccatcgacctgcggaccaccgtggaggtatcccgtaaaagccgtggcgtccacattcactcggccttcaaggacgagccggataagaacctgtatatcaagacggccgttaaaaggatgcagaagtgtgcggatatccggaacgtgaacatcgctgtttcctcgaggatccccgtggcttcgggcctgggctcctcggctgccgtgaccgtcgccaccatcggcgccttgaacgaggagttcagcgccggcttgagtaaaaaagacatcgcgtatatggcctaccagacagagctggaagtgcagggagcggcgagcccgaccgatacgttcgtgtccaccatgggcgggaccgttgtcgtgcccgacatgaggacactgccgcctatcacctgcggcatcgtggtcggccacaccggcatatcaaaatccacttcacgcatggtatcccgggtgagaacgctcaaagaaaaatacccggacgtcatcgacggcatcatggactcgatcggggatatctccgcgcggggcgaggatttaataaaacagaacgactatcgttccataggcgagctcatgaacgtcaaccaggggctgctggacgccctgggcatcaccatacccgagctctcactccagatatatgccgcacgccagcacggcgcctacggcgccaagatcaccggcgcgggcggagggggatgcatggtggccatatgcgatgacaaaaattgtaaagagatcgcgaccgccatcgggcgctcatacggcgacagcttcatcagcaagccaacggcggagggaatattcatccaatga

SEQ ID NO: 23 - DNA, 70 bp, Artificial Sequence
  Synthetic sequence, ampH-attL-phi80
atgcgcactccttacgtactggctctactggtttctttgcgaaaggtcatttttcctgaatatgctcaca

SEQ ID NO: 24 - DNA, 64 bp, Artificial Sequence
  Synthetic sequence, ampH-attR-phi80
ttaaggaatcgcctggaccatcatcggcgagccgttctgacgtttgttgacagctggtccaatg

SEQ ID NO: 25 - DNA, 68 bp, Artificial Sequence
  Synthetic sequence, DampC-phL
ctgatgaactgtcacctgaatgagtgctgatgaaaatatagaaaggtcatttttcctgaatatgctca

SEQ ID NO: 26 - DNA, 64 bp, Artificial Sequence
  Synthetic sequence, DampC-phR
attcgccagcataacgatgccgctgttgagctgaggaacacgtttgttgacagctggtccaatg

SEQ ID NO: 27 - DNA, 18 bp, Artificial Sequence
  Synthetic sequence, ampH-t1
gcgaagccctctccgttg

SEQ ID NO: 28 - DNA, 21 bp, Artificial Sequence
  Synthetic sequence, ampH-t2
agccagtcagcctcatcagcg

SEQ ID NO: 29 - DNA, 21 bp, Artificial Sequence
  Synthetic sequence, ampC-t1
gattcccacttcaccgagccg

SEQ ID NO: 30 - DNA, 21 bp, Artificial Sequence
  Synthetic sequence, ampC-t2
ggcaggtatggtgctctgacg

SEQ ID NO: 31 - DNA, 64 bp, Artificial Sequence
  Synthetic sequence, crtE-attRphi80
atgacggtctgcgcaaaaaaacacgttcatctcactcgcgcgtttgttgacagctggtccaatg

SEQ ID NO: 32 - DNA, 68 bp, Artificial Sequence
  Synthetic sequence, crtZ-attLphi80
atgttgtggatttggaatgccctgatcgttttcgttaccggaaaggtcatttttcctgaatatgctca

SEQ ID NO: 33 - DNA, 21 bp, Artificial Sequence
  Synthetic sequence, crtZ-test
ccgtgtggttctgaaagccga

SEQ ID NO: 34 - DNA, 21 bp, Artificial Sequence
  Synthetic sequence, crtE-test
cgttgccgtaaatgtatccgt

(19) United States
(12) Patent Application Publication
(10) Pub. No.: US 2018/0291127 A1
(43) Pub. Date: Oct. 11, 2018

(54) METHOD FOR PRODUCING POLYISOPRENE

(71) Applicants: BRIDGESTONE CORPORATION, Tokyo (JP); Ajinomoto Co., Inc., Tokyo (JP)

(72) Inventors: Fumiaki NISHIURA, Mitaka-shi (JP); Yosuke NISHIO, Kawasaki-shi (JP); Hiroaki RACHI, Kawasaki-shi (JP); Satoshi HIOKI, Kawasaki-shi (JP)

(73) Assignees: BRIDGESTONE CORPORATION, Tokyo (JP); Ajinomoto Co., Inc., Tokyo (JP)

(21) Appl. No.: 15/569,862

(22) PCT Filed: Apr. 28, 2016
(86) PCT No.: PCT/JP2016/002240

§ 371 Date: Oct. 27, 2017

(30) Foreign Application Priority Data
Apr. 30, 2015 (JP) 2015-093585

(51) Int. Cl. C08F 36/08; C12P 5/02

(52) U.S. Cl. CPC C08F 36/08; B60C 1/00; C12P 5/026

(57) ABSTRACT

A method for producing polyisoprene comprising: polymerizing isoprene by using an isoprene-containing composition containing isoprene and an oxygen-containing neutral compound, and a polymerization catalyst composition containing a rare earth element compound and an organometallic compound, wherein: —a compounding amount of the organometallic compound is 0.6 to 3.0 parts by mass per 100 parts by mass of the isoprene-containing composition.

### TECHNICAL FIELD

This disclosure relates to a method for producing polyisoprene.

### BACKGROUND

A rubber product such as tire is requested of excellent breaking resistance, wear resistance, crack growth resistance, etc., and thus a rubber component having excellent elasticity is necessarily used as its raw material Known as the rubber component is a natural rubber collected from a rubber tree, which mainly contains polyisoprene with a high molecular weight.

Recently, natural rubber is in high demand, while on the other hand, along with the reduction of rubber tree resource, the price of natural rubber is rising. Therefore, preparation of a polyisoprene with properties identical to a polyisoprene of rubber tree derived natural rubber is being studied widely.

Known as the polyisoprene instead of the polyisoprene of natural rubber are synthesized polyisoprene prepared with a chemical method such as distillation of petroleum, and fermented polyisoprene prepared with a method using microbes (see PTL1 to PTL3).

### CITATION LIST

### Patent Literature

PTL1: JP2011505841A

PTL2: JP2011518564A

PTL3: JP2013514375A

### SUMMARY

### Technical Problem

However, an isoprene-containing composition using an isoprene prepared with the aforementioned conventional methods, particularly in the case with existence of impurities inhibiting the polymerization of the isoprene, there was a risk of deterioration of the catalytic activity, which disables production of a polyisoprene with sufficiently high molecular weight.

Then, this disclosure aims to provide a method for producing polyisoprene, which, even in the case with existence of impurities other than isoprene inhibiting the polymerization of isoprene, is capable of controlling its type and amount, and controlling the type and among of the polymerization catalyst, to thereby produce a polyisoprene with a high molecular weight as compared to the case of polymerizing a highly purified isoprene.

### Solution to Problem

The gist of the present disclosure is as follows.

The method for producing polyisoprene of this disclosure is a method for producing polyisoprene comprising: polymerizing isoprene by using an isoprene-containing composition containing isoprene and an oxygen-containing neutral compound, and a polymerization catalyst composition containing a rare earth element compound and an organometallic compound, wherein:

a compounding amount of the organometallic compound is 0.6 to 3.0 parts by mass per 100 parts by mass of the isoprene-containing composition.

In the method for producing polyisoprene of this disclosure, it is preferable that a compounding amount of the oxygen-containing neutral compound is 0.05 to 1.0 parts by mass per 100 parts by mass of the isoprene-containing composition.

Moreover, in the method for producing polyisoprene of this disclosure, the isoprene-containing composition may be prepared via a fermentation method.

Further, in the method for producing polyisoprene of this disclosure, it is preferable that the oxygen-containing neutral compound is selected from the group consisting of an ether, an ester and a ketone.

Further, in the method for producing polyisoprene of this disclosure, it is preferable that a number-average molecular weight (Mn) of the polyisoprene is 1,000,000 or more.

Further, in the method for producing polyisoprene of this disclosure, it is preferable that the number-average molecular weight (Mn) of the polyisoprene is 1,300,000 or more.

Further, in the method for producing polyisoprene of this disclosure, it is preferable that a molecular weight distribution (Mw/Mn) of the polyisoprene is 2.5 or less.

Further, in the method for producing polyisoprene of this disclosure, it is preferable that a cis-1,4-bond content of the polyisoprene is 98% or more.

Further, in the method for producing polyisoprene of this disclosure, it is preferable that the rare earth element compound is a rare earth element-containing compound or a reaction product of the rare earth element-containing compound and a Lewis base, the rare earth element-containing compound or reaction product having metal-nitrogen bond (M-N bond), and the rare earth element-containing compound is a compound containing scandium, yttrium or a lanthanoid element selected from elements of atomic numbers 57 to 71.

Further, in the method for producing polyisoprene of this disclosure, it is preferable that the organometallic compound is a compound represented with

the following formula (S2)

YR⁴ₐR⁵_{b}R⁶_{c} (S2)

where Y is a metallic element selected from the group consisting of Group 1, Group 2, Group 12 and Group 13; R⁴ and R⁵ are C1 to C10 hydrocarbon group or hydrogen atom, and may be either identical or different; R⁶ is a C1 to C10 hydrocarbon group; R⁶ may be either identical to or different from the R⁴ or R⁵; if Y is a Group 1 metallic element, a is 1, and b, c are 0; if Y is a Group 2 metallic element or a Group 12 metallic element, a and b are 1, and c is 0; and if Y is a Group 13 metallic element, a, b and c are 1; or

the following formula (S3)

AlR⁷R⁸R⁹ (S3)

where R⁷ and R⁸ are C1 to C10 hydrocarbon group or hydrogen atom, and may be either identical or different; R⁹ is a C1 to C10 hydrocarbon group; and R⁹ may be either identical to or different from the R⁷ or R⁸.

Further, in the method for producing polyisoprene of this disclosure, it is preferable that the polymerization catalyst composition is at least one compound selected from the group consisting of an aluminoxane compound, a halogen compound, an ionic compound, and a compound capable of serving as an anionic ligand.

### Advantageous Effect

According to the method for producing polyisoprene of this disclosure, it is possible to produce a polyisoprene having a comparatively high molecular weight, and to thereby improve the produced synthesized polyisoprene.

### BRIEF DESCRIPTION OF THE DRAWINGS

FIG. 1 illustrates a pAH162-P^{ara}-mvaES plasmid constructed in the examples;

FIG. 2A illustrates a pAH162-λattL-Tc^{R}-ΔattR integrative vector, and FIG. 2B illustrates the pAH162-ΔattL-Km^{R}-ΔattR integrative vector constructed in the examples;

FIG. 3 illustrates a pAH162-P_{tac} integrative expression vector used in the examples;

FIG. 4 illustrates a KDyI operon with a chemically synthesized codon optimized;

FIG. 5A illustrates a plasmid pAH162-Tc-P_{tac}-KDyI constructed in the examples for holding a KDyI operon with an optimized codon, and FIG. 5B illustrates a plasmid pAH162-Km-P_{tac}-KDyI constructed in the examples for holding a KDyI operon with an optimized codon;

FIG. 6 illustrates an integrative plasmid pAH162-P_{tac}-mvk constructed in the examples for holding an mvk gene derived from M. paludicola;

FIG. 7A illustrates a modified genome ΔampC::attBₚₕᵢ₈₀ constructed in the examples; FIG. 7B illustrates a modified genome ΔampH::attBₚₕᵢ₈₀ constructed in the examples; and FIG. 7C illustrates a modified genome Δcrt::attBₚₕᵢ₈₀ constructed in the examples;

FIG. 8A illustrates a modified genome Δcrt::pAH162-P_{tac}-mvk constructed in the examples, and FIG. 8B illustrates a modified genome Δcrt::P_{tac}-mvk constructed in the examples;

FIG. 9A illustrates a modified genome ΔampH::pAH162-Km-P_{tac}-KDyI constructed in the examples, and FIG. 9B illustrates a modified genome ΔampC::pAH162-Km-P_{tac}-KDyI constructed in the examples;

FIG. 10A illustrates a modified genome ΔampH::pAH162-Pₐᵣₐ-mvaES constructed in the examples, and FIG. 10B illustrates a modified genome ΔampC::pAH162-Pₐᵣₐ-mvaES constructed in the examples; and

FIG. 11 illustrates an outline of an isoprene recovery apparatus used in the examples.

### DETAILED DESCRIPTION

(Method for Producing Polyisoprene)

An embodiment for the method for producing polyisoprene of this disclosure is described in detail hereinafter.

The method for producing polyisoprene of an example of this disclosure (referred to as “the production method of the example” as well hereinafter) is a method for polymerizing isoprene by using: an isoprene-containing composition containing isoprene and an oxygen-containing neutral compound, and a polymerization catalyst composition containing a rare earth element compound and an organometallic compound.

—Isoprene-Containing Composition—

The isoprene-containing composition used in the method for producing polyisoprene of the examples of this disclosure (referred to as “the isoprene-containing composition” as well hereinafter) contains isoprene and an oxygen-containing neutral compound.

Here, the oxygen-containing neutral compound is considered as coordinated to a central metal of a metallic catalyst used in the polymerization of isoprene, thereby improving the catalytic activity. According to the method for producing polyisoprene using this isoprene-containing composition, it is possible to produce a polyisoprene with a comparatively high molecular weight.

—Isoprene—

The isoprene contained in the isoprene-containing composition is not specifically limited, and may be either an isoprene obtainable with a chemical method (synthesized isoprene) or an isoprene prepared with a method using microbes (fermented isoprene).

The synthesized isoprene may be obtained as a C₅ fraction in fractional distillation of petroleum.

The fermented isoprene may be obtained by, for example, the fermentation mentioned below, and in this case, the product of fermentation method may be used directly as the isoprene-containing composition.

It is preferable that the synthesized isoprene and the fermented isoprene are purified before used in the polymerization reaction, where the necessity is higher as for the fermented isoprene.

Examples of the method for purifying the fermented isoprene include column chromatography, distillation, etc., and in particular, from the viewpoint of simple convenience and economy, column chromatography is preferable.

In the case of column chromatography, examples of a filler of the column (solid) include activated alumina, silica gel, molecular sieve, reduced copper, etc., and in particular, activated alumina is preferable, and examples of an elute (liquid) include hydrocarbons, etc., and in particular, hexane is preferable.

Here, from the viewpoint of sufficiently removing impurities inhibiting the polymerization of the isoprene, a weight ratio of the filler with respect to isoprene is preferably 0.1 to 2.0, more preferably 0.5 to 1.0. A purification time is preferably 2 to 8 hours, more preferably 4 to 6 hours.

—Oxygen-Containing Neutral Compound—

Examples of the oxygen-containing neutral compound include ether, ester, ketone, etc.

Specific examples of the ester include ethyl acetate, methyl acetate, etc., and specific examples of the ketone include methyl vinyl ketone, methyl ethyl ketone (2-butanone), diacetyl(2,3-butanedione), methyl isobutyl ketone, etc. In particular, ethyl acetate and 3-methylfuran are preferable.

By selecting the aforementioned compounds as the oxygen-containing neutral compound, it is possible to improve the effect of the method for producing polyisoprene to produce a polyisoprene having a comparatively high molecular weight.

Note that the isoprene-containing composition may contain components other than the aforementioned isoprene and oxygen-containing neutral compound.

Moreover, the isoprene-containing composition refers to one without containing a solvent, and may be, for example, one consisting of merely the aforementioned isoprene and oxygen-containing neutral compound.

According to the method for producing polyisoprene using the aforementioned isoprene-containing composition, it is possible to produce a polyisoprene having a comparatively high molecular weight with existence of an oxygen-containing neutral compound other than isoprene. Thereby, it is possible to improve the strength of the produced synthesized polyisoprene.

Here, the isoprene-containing composition is an isoprene-containing composition of which a weight of isoprene is 98.5 mass % or more. From the viewpoint of sufficiently obtaining the effect of this disclosure, this ratio is preferably 98.8 mass % or more, further preferably 99.0 mass % or more.

Here, from the viewpoint of sufficiently obtaining the aforementioned effect of the method for manufacturing polyisoprene, i.e., the capacity of producing a polyisoprene having a comparatively high molecular weight, the compounding amount of the oxygen-containing neutral compound is 0.05 parts by mass or more, preferably 0.07 parts by mass or more, particularly preferably 0.1 parts by mass or more per 100 parts by mass of isoprene. Moreover, from the viewpoint of reducing the risk that the the oxygen-containing neutral compound deteriorates the catalytic activity in contrary, the compounding amount of the oxygen-containing neutral compound is preferably 1.0 part by mass or less, more preferably 0.7 parts by mass or less, particularly preferably 0.5 parts by mass or less.

The fermentation method for producing the aforementioned fermented isoprene is described hereinafter.

In the fermentation method, a gene encoding isoprene synthase is transduced into a host cell, thereby transforming the host cell, to culture this transformant and produce isoprene.

In the method according to the example according to the fermentation method, from the viewpoint of increasing the production volume of isoprene, in addition to the gene encoding isoprene synthase, it is preferable to produce the transformant by further transducing genes encoding enzymes involved in a mevalonic acid (MVA) pathway and/or a methyl erythritol phosphate (MEP) pathway involved in synthesis of dimethylallyl diphosphate (DMAPP), which is a substrate of isoprene synthase.

Further, in the method according to the example, from the viewpoint of increasing the production volume of isoprene, it is preferable to produce the transformant by further transducing a gene encoding an isopentenyl-diphosphate delta isomerase having a capacity of converting isopentenyl diphosphate (IPP) into dimethylallyl diphosphate (DMAPP).

The enzyme expressed in the transformant in the fermentation method may be either identical to or different from the species of host cell.

As an example of this disclosure, it is preferable to use Pantoea ananatis as a host cell, one derived from Mucuna as an isoprene synthase, one derived from Saccharomyces cerevisiae, Enterococcus faecalis or Metanocella pardicola as an enzyme involved in the mevalonic acid pathway, and one derived from Saccharomyces cerevisiae as an isopentenyl-diphosphate delta isomerase. According to this combination, isoprene may be produced at a high efficiency.

[Host]

A host used in the method according to the example may be any cell without being limited, as long as containing genes encoding enzymes involved in a mevalonic acid (MVA) pathway and/or a methyl erythritol phosphate (MEP) pathway involved in synthesis of dimethylallyl diphosphate (DMAPP), which is a substrate of isoprene synthase. The host is preferably a bacterium or a fungus.

The bacterium may be either a gram-positive bacterium or a gram-negative bacterium.

Examples of the gram-positive bacterium include bacteria belonging to the genera Bacillus, Listeria, Staphylococcus, Streptococcus, Enterococcus, Clostridium, Corynebacterium and Streptomyces. Bacteria belonging to the genera Bacillus and Corynebacterium are preferable.

Examples of the bacteria belonging to the genus Bacillus may include Bacillus subtilis, Bacillus anthracis, and Bacillus cereus. Bacillus subtilis is more preferable. Examples of the bacteria belonging to the genus Corynebacterium may include Corynebacterium glutamicum, Corynebacterium efficiens, and Corynebacterium callunae. Corynebacterium glutamicum is more preferable.

Examples of the gram-negative bacterium may include bacteria belonging to the genera Escherichia, Pantoea, Salmonella, Vivrio, Serratia, and Enterobacter. The bacteria belonging to the genera Escherichia, Pantoea and Enterobacter are preferable.

Escherichia coli is preferable as the bacteria belonging to the genus Escherichia.

Examples for a strain of Escherichia coli (E. coli) include well-known ones in the art, for example, DH5α, JM109, etc.

Examples of the bacteria belonging to the genus Pantoea may include Pantoea ananatis, Pantoea stewartii, Pantoea agglomerans, and Pantoea citrea. Pantoea ananatis and Pantoea citrea are preferable. Strains exemplified in EP0952221, which is incorporated herein by reference in its entirety, may be used as the bacteria belonging to the genus Pantoea. Examples of representative strains of the bacteria belonging to the genus Pantoea may include Pantoea ananatis AJ13355 strain (FERM BP-6614) and Pantoea ananatis AJ13356 strain (FERM BP-6615), both of which are disclosed in EP0952221, which is incorporated herein by reference in its entirety, and Pantoea ananatis SC17(0) strain. Pantoea ananatis SC17(0) was deposited to Russian National Collection of Industrial Microorganisms (VKPM), GNII Genetika (address: Russia, 117545 Moscow, 1 Dorozhny proezd. 1) as of Sep. 21, 2005, with the deposit number of VKPM B-9246.

Examples of the bacteria belonging to the genus Enterobacter may include Enterobacter agglomerans and Enterobacter aerogenes. Enterobacter aerogenes is preferable. The bacterial strains exemplified in EP0952221, which is incorporated herein by reference in its entirety, may be used as the bacteria belonging to the genus Enterobacter. Examples of representative strains of the bacteria belonging to the genus Enterobacter may include Enterobacter agglomerans ATCC 12287 strain, Enterobacter aerogenes ATCC 13048 strain, Enterobacter aerogenes NBRC12010 strain (Biotechnol. Bioeng., 2007 Mar. 27; 98(2): 340-348, which is incorporated herein by reference in its entirety), and Enterobacter aerogenes AJ110637 (FERM BP-10955). The Enterobacter aerogenes AJ110637 strain was deposited to International Patent Organism Depositary (IPOD), National Institute of Advanced Industrial Science and Technology (AIST) (Chuo No. 6. Higashi 1-1-1, Tsukuba City, Ibaraki Pref., JP, Postal code 305-8566) as of Aug. 22, 2007, with the deposit number of FERM P-21348 and was transferred to the international deposition based on Budapest Treaty on Mar. 13, 2008, and the receipt number FERM BP-10955 was given thereto.

Examples of the fungus may include microorganisms belonging to the genera Saccharomyces, Schizosaccharomyces, Yarrowia, Trichoderma, Aspergillus, Fusarium, and Mucor. The microorganisms belonging to the genera Saccharomyces, Schizosaccharomyces, Yarrowia, or Trichoderma are preferable.

Examples of the microorganisms belonging to the genus Saccharomyces may include Saccharomyces carlsbergensis, Saccharomyces cerevisiae, Saccharomyces diastaticus, Saccharomyces douglasii, Saccharomyces kluyveri, Saccharomyces norbensis, and Saccharomyces oviformis. Saccharomyces cerevisiae is preferable.

Schizosaccharomyces pombe is preferable as a microorganism belonging to the genus Schizosaccharomyces.

Yarrowia lypolytica is preferable as a microorganism belonging to the genus Yarrowia.

Examples of the microorganisms belonging to the genus Trichoderma may include Trichoderma harzianum, Trichoderma koningii, Trichoderma longibrachiatum, Trichoderma reesei, and Trichoderma viride. Trichoderma reesei is preferable.

Other than the bacteria and the fungi mentioned above, examples of the host include an insect cell, an animal cell, a plant cell, a bacterial cell, etc.

Note that such host may be used alone or in a combination of two or more.

[Isoprene Synthase]

Examples of the isoprene synthase used in the method according to the example include isoprene synthase derived from kudzu (Pueraria montana) (IspK), isoprene synthase derived from poplar, isoprene synthase derived from Mucuna (Mucuna pruriens) (IspM) (US20140113344A1), isoprene synthase derived from willow (Salix), isoprene synthase derived from false acacia (Robinia pseudoacacia), isoprene synthase derived from wisteria (Wisterria), isoprene synthase derived from eucalyptus (Eucalyptus globulus), isoprene synthase derived from tea tree (Melaleuca alterniflora), etc. (see, e.g., Evolution 67 (4), 1026-1040 (2013) which is incorporated herein by reference in its entirety). In a preferable embodiment, the isoprene synthase may be derived from kudzu; in another preferable embodiment, the isoprene synthase may be derived from poplar; and in still another preferable embodiment, the isoprene synthase may be derived from Mucuna.

Note that such isoprene synthase may be used alone or in a combination of two or more.

[Enzyme Involved in Mevalonic Acid (MVA) Pathway]

Examples of the enzymes involved in the mevalonate (MVA) pathway may include mevalonate kinase (EC: 2.7.1.36; example 1, Erg12p, ACCESSION ID NP_013935; example 2, AT5G27450, ACCESSION ID NP_001190411), phosphomevalonate kinase (EC: 2.7.4.2; example 1, Erg8p, ACCESSION ID NP_013947; example 2, AT1G31910, ACCESSION ID NP_001185124), diphosphomevalonate decarboxylase (EC: 4.1.1.33; example 1, Mvd1p, ACCESSION ID NP_014441; example 2, AT2G38700, ACCESSION ID NP_181404; example 3, AT3G54250, ACCESSION ID NP_566995), acetyl-CoA-C-acetyltransferase (EC: 2.3.1.9; example 1, Erg10p, ACCESSION ID NP_015297; example 2, AT5G47720, ACCESSION ID NP_001032028; example 3, AT5G48230, ACCESSION ID NP_568694), hydroxymethylglutaryl-CoA synthase (EC: 2.3.3.10; example 1, Erg13p, ACCESSION ID NP_013580; example 2, AT4G11820, ACCESSION ID NP_192919; example 3, MvaS, ACCESSION ID AAG02438), hydroxymethylglutaryl-CoA reductase (EC: 1.1.1.34; example 1, Hmg2p, ACCESSION ID NP_013555; example 2, Hmg1p, ACCESSION ID NP_013636; example 3, AT1G76490, ACCESSION ID NP_177775; example 4, AT2G17370, ACCESSION ID NP_179329, EC: 1.1.1.88, example, MvaA, ACCESSION ID P13702), and acetyl-CoA-C-acetyltransferase/hydroxymethylglutaryl-CoA reductase (EC: 2.3.1.9/1.1.1.34, example, MvaE, ACCESSION ID AAG02439).

Examples of the enzymes involved in the mevalonate (MVA) pathway may include those expressed from genes derived from microorganisms belonging to the genus Methanosarcina such as Methanosarcina mazei, the genus Methanocella such as Methanocella paludicola, the genus Corynebacterium such as Corynebacterium variabile, the genus Methanosaeta such as Methanosaeta concilii, and the genus Nitrosopumilus such as Nitrosopumilus maritimus.

Note that such enzyme involved in the mevalonic acid (MVA) pathway may be used alone or in a combination of two or more.

[Enzyme Involved in the Methylerythritol Phosphate (MEP) Pathway]

Examples of the enzymes involved in the methylerythritol phosphate (MEP) pathway may include 1-deoxy-D-xylulose-5-phosphate synthase (which synthesizes 1-deoxy-D-xylose-5-phosphate from a pyruvate and D-glyceraldehyde-3-phosphate) (EC: 2.2.1.7, example 1, Dxs, ACCESSION ID NP_414954; example 2, AT3G21500, ACCESSION ID NP_566686; example 3, AT4G15560, ACCESSION ID NP_193291; example 4, AT5G11380, ACCESSION ID NP_001078570), 1-deoxy-D-xylulose-5-phosphate reductoisomerase (EC: 1.1.1.267; example 1, Dxr, ACCESSION ID NP_414715; example 2, AT5G62790, ACCESSION ID NP_001190600), 4-diphosphocytidyl-2-C-methyl-D-erythritol synthase (EC: 2.7.7.60; example 1, IspD, ACCESSION ID NP_417227; example 2, AT2G02500, ACCESSION ID NP_565286), 4-diphosphocytidyl-2-C-methyl-D-erythritol kinase (EC: 2.7.1.148; example 1, IspE, ACCESSION ID NP_415726; example 2, AT2G26930, ACCESSION ID NP_180261), 2-C-methyl-D-erythritol-2,4-cyclodiphosphate synthase (EC: 4.6.1.12; example 1, IspF, ACCESSION ID NP_417226; example 2, AT1G63970, ACCESSION ID NP_564819), 1-hydroxy-2-methyl-2-(E)-butenyl-4-diphosphate synthase (EC: 1.17.7.1; example 1, IspG, ACCESSION ID NP_417010; example 2, AT5G60600, ACCESSION ID NP_001119467), and 4-hydroxy-3-methyl-2-butenyl diphosphate reductase (EC: 1.17.1.2; example 1, IspH, ACCESSION ID NP_414570; example 2, AT4G34350, ACCESSION ID NP_567965).

The MEP pathway is known as widely existing in prokaryotic microorganisms and plants, and enzymes derived from these creatures may be used (Eisenreich W et al., Biosynthesis of isoprenoids via the non-mevalonate pathway, Cell Mol Life Sci. 61, 1401-1426, 2004). Examples thereof include those expressed from genes derived from Escherichia coli, Pantoea ananatis, Corynebacterium glutamicum, creatures belonging to the genus Enterobacter, etc.

Note that the aforementioned enzymes involved in the methylerythritol phosphate (MEP) pathway may be used alone or in a combination of two or more.

[Isopentenyl-Diphosphate Delta Isomerase]

Examples of isopentenyl-diphosphate delta isomerase (isopentenyl-diphosphate isomerase) may include Idi1p (ACCESSION ID NP_015208), AT3G02780 (ACCESSION ID NP_186927), AT5G16440 (ACCESSION ID NP_197148) and Idi (ACCESSION ID NP_417365).

In the fermentation method used in this disclosure, enzymes other than the aforementioned enzymes and proteins other than enzymes may be used as necessary.

As long as having the functions of the aforementioned enzymes or proteins, modified products of the aforementioned enzymes or proteins may be used, without being limited to the amino acid sequence of the aforementioned enzymes or proteins. Examples for the modified products include those having substitution, deletion, insertion, addition and the like of one or more amino acids.

Note that, although varying depending on the types of amino acid residual groups related to the substitution, deletion, insertion, addition and the like, and the position in the three-dimensional structure of the protein of the amino acid residual group, “one or more” refers to 1 to 10, preferably 1 to 5, more preferably 1 to 3, further more preferably 1 to 2.

Specific examples of those having substitution include those having conservative substitution.

“Conservative substitution” refers to substitution between amino acids having similar properties, and specifically refers to substitution between Phe, Trp or Tyr in the case of aromatic amino acids, substitution between Leu, Ile or Val in the case of hydrophobic amino acids, substitution between Gln and Asn in the case of polar amino acids, substitution between Lys, Arg or His in the case of basic amino acids, substitution between Asp and Glu in the case of acidic amino acids, and substitution between Ser and Thr in the case of amino acids having hydroxyl groups.

Specific examples of substitution treated as conservative substitution include substitution from Ala to Ser or Thr; substitution from Arg to Gln, His or Lys; substitution from Asn to Glu, Gln, Lys, His or Asp; substitution from Asp to Asn, Glu or Gln; substitution from Cys to Ser or Ala; substitution from Gln to Asn, Glu, Lys, His, Asp or Arg; substitution from Glu to Gly, Asn, Gln, Lys or Asp; substitution from Gly to Pro; substitution from His to Asn, Lys, Gln, Arg or Tyr; substitution from Ile to Leu, Met, Val or Phe; substitution from Leu to Ile, Met, Val or Phe; substitution from Lys to Asn, Glu, Gln, His or Arg; substitution from Met to Ile, Leu, Val or Phe; substitution from Phe to Trp, Tyr, Met, Ile or Leu; substitution from Ser to Thr or Ala; substitution from Thr to Ser or Ala; substitution from Trp to Phe or Tyr; substitution from Tyr to His, Phe or Trp; and substitution from Val to Met, Ile or Leu.

Base sequences (DNA sequences and RNA sequences) of the genes encoding the aforementioned enzymes or proteins are well known in the art.

Such base sequence is not limited to the aforementioned base sequences as long as having the functions of the enzyme or protein encoded in its base sequence, and modified products of the aforementioned base sequence may be used.

The modified products may be either obtained via naturally occurring mutantation or obtained via artificial mutantation (e.g., by transducing a variation from/into the sequence by a site-specific mutagenesis).

An identity of the modified product of the base sequence to the original base sequence is 80% or more, preferably 90% or more, further preferably 95% or more, further more preferably 97% or more.

The modified product of the base sequence, as long as having the functions of the enzyme or protein encoded in the original base sequence, may be a base sequence complementary to a base sequence well-known in the art, which hybridizes with the base sequence under a stringent condition, or a probe made from a part thereof.

The “stringent condition” refers to a condition where a specific hybridization occurs whereas a non-specific hybridization does not occur. For example, such condition is a condition where DNAs having a high sequence identity, e.g., a sequence identity of 80% or more, preferably 90% or more, more preferably 95% or more, further more preferably 97% or more, hybridize with each other, whereas DNAs having a low sequence identity, e.g., a sequence identity of less than 80%, do not hybridize with each other; or, a condition where hybridization may be maintained after one time, preferably 2 to 3 times of washing under a condition of 60° C., 1×SSC, 0.1% SDS, preferably a condition of 60° C., 0.1×SSC, 0.1% SDS, more preferably 68° C., 0.1×SSC, 0.1%, which is an ordinary condition for washing operation in Southern hybridization experiment.

A codon encoding the amino acids of the aforementioned enzyme or protein may vary depending on a usage frequency of the codon in a host for expression of the enzyme or protein, thereby varying the base sequence of the gene encoding the aforementioned enzyme or protein.

[Method for Transducing Gene]

Examples of the method for transducing gen used in the method according to the example include methods well-known in the art.

Such methods include methods for transducing into a host cell a plasmid vector having a gene encoding a target protein such as enzyme and the like, via a method using microbial cells (competent cells) treated with calcium chloride, electroporation method, etc.; methods for transducing into a chromosome of a bacterium a phage vector with a gene encoding a target protein transduced therein by infecting microbial cells of the bacterium with the same; and the like.

It is preferable that in the gene transduction in this disclosure, a copy number of a target gene on a chromosome is increased to enhance an activity of the target protein. In the case of transducing a gene of an enzyme which is not originally possessed by a microbe as the host, it is preferable that the microbe contains 1 copy or more of the gene on its chromosome. Moreover, in the case of transducing a gene of an enzyme which is possessed by the microbe as the host, it is preferable that the microbe has a plurality of copies, preferably 2 copies or more, more preferably 3 copies or more of the gene on its chromosome.

In the gene transduction in this disclosure, the transduction of the target gene may also be achieved by transducing a plasmid carrying the gene on the host cell.

Here, increase of the copy number can also be accomplished by utilizing transposon or Mu phage to transfer the target gene onto the genome of the host.

Here, examples of the vector include a plasmid vector, a viral vector, etc., without being limited thereto. The vector may be either a DNA vector or an RNA vector. Examples of the vector include a monocistronic vector, a bicistronic vector, a polycistronic vector, etc.

The vector may be appropriately selected depending on the used host. Examples of the expression vector may include ColE-based plasmids typified by pBR322 derivatives, pACYC-based plasmids having a p15A origin, pSC-based plasmids, and mini F plasmids derived from an F factor of Bac and the like in Escherichia coli (E. coli).

Construction of the vector may be performed with a genetic engineering method such as PCR method, crossover PCR method, In-fusion method, λ-Red method, etc., by appropriately using an enzyme such as a restriction enzyme, a DNA polymerase, a ligase, etc.

Note that it is preferable that a system used for the aforementioned gene transduction is constructed with an ordinary method so as to be capable of inducing the expression of the target gene in the cultivation described below. Examples of such induction include IPTG induction, arabinose induction, etc. Here, examples of a promoter used in the vector include a tryptophan promoter such as trc, tac and the like, lac promoter, T7 promoter, T5 promoter, T3 promoter, SP6 promoter, arabinose-inducible promoter, cold-shock promoter, tetracycline-inducible promoter, etc.

Note that in this disclosure, it is preferable to transduce the isoprene synthase into a cytoplasm of Pantoea ananatis with the aforementioned vector, and to further perform chromosome fixation with enzymes involved in an upstream of the mevalonic acid pathway as artificial operons, and to perform chromosome fixation to mevalonate kinase independently with other enzymes as artificial operons among enzymes involved in a downstream of the mevalonic acid pathway, to thereby fix the same to a chromosome of Pantoea ananatis.

Details of transformant cultivation would be described in the following.

### [Cultivation Method]

Examples of methods of culturing the transformant include batch cultivation, feeding cultivation (fed batch cultivation), continuous cultivation (perfusion cultivation), etc. In particular, from the viewpoint of the capability of industrial production of isoprene, feeding cultivation and continuous cultivation are preferable.

Batch cultivation is a method in which the cultivation is performed by preparing a new culture medium in each time of cultivation, and adding the transformant therein, without adding the culture medium, etc. In batch cultivation, the transformant follows a growth cycle including a lag phase, a logarithmic growth phase and a stationary phase, where the production volume of isoprene increases in the logarithmic growth phase and the stationary phase.

Feeding cultivation is a method in which the transformant is added into a culture medium to perform cultivation, and the culture medium and components of the culture medium are appropriately added during the cultivation. In feeding cultivation, it is possible to optimize a growth rate by adjusting a cell density, or to maintain a productivity of isoprene by diluting harmful substances accumulated in the culture medium.

The continuous cultivation method is a method in which a certain amount of the medium is continuously supplied to a culture tank at a constant rate while the same amount of the medium is removed. In continuous cultivation, a constant culture environment may be maintained. More specifically, according to this cultivation, it is possible to maintain the logarithmic growth phase mainly with respect to the transformant in the culture medium, and thus the productivity of isoprene may be easily maintained. Moreover, accumulation of metabolic byproducts and dead cells, which potentially have adverse effects on the growth of the transformant, can be prevented.

[Culture Condition]

A culture condition is not particularly limited as long as capable of expressing the protein of this disclosure, and a standard cell culture condition can be used.

The cultivation may be performed under an aerobic, oxygen-free, or anaerobic condition depending on a nature of the host cell.

The culture temperature is preferably 20 to 37° C., the culture atmosphere is preferably about 6% to about 84% of CO₂ concentration, and the pH value is preferably about 5 to about 9.

[Culture Medium]

The culture medium used in the method according to the example is either a natural medium or a synthesized medium, and is a culture medium containing at least a carbon source for forming an isoprene skeleton, and containing optionally a nitrogen source, an organic trace nutrient source, an inorganic matter (mineral), etc.

The carbon source may include carbohydrates such as monosaccharides, disaccharides, oligosaccharides, polysaccharides and invert sugars; compounds having one carbon atom (hereinafter referred to as a C1 compound); lipids (oils, etc.); fatty acids; glycerine fatty acid esters; polypeptides; renewable carbon sources; yeast extracts; phospholipids; glycerolipids; glycerol; acetate, etc.

Such carbon sources may be used alone or in a combination of two or more.

The carbon sources would be described in details in the following.

Examples of the monosaccharides among the carbohydrates may include triose such as ketotriose (dihydroxyacetone) and aldotriose (glyceraldehyde); tetrose such as ketotetrose (erythrulose) and aldotetrose (erythrose, threose); pentose such as ketopentose (ribulose, xylulose), aldopentose (ribose, arabinose, xylose, lyxose) and deoxysaccharide (deoxyribose); hexose such as ketohexose (psychose, fructose, sorbose, tagatose), aldohexose (allose, altrose, glucose, mannose, gulose, idose, galactose, tallose), and deoxysaccharide (fucose, fucrose, rhamnose); and heptose such as sedoheptulose. C6 sugars such as fructose, mannose, galactose and glucose; and C5 sugars such as xylose and arabinose are preferable.

Examples of the disaccharides may include sucrose, lactose, maltose, trehalose, turanose, and cellobiose. Sucrose and lactose are preferable.

Examples of the oligosaccharides may include trisaccharides such as raffinose, melezitose and maltotriose; tetrasaccharides such as acarbose and stachyose; and other oligosaccharides such as fructooligosaccharide (FOS), galactooligosaccharide (GOS) and mannan-oligosaccharide (MOS).

Examples of the polysaccharides may include glycogen, starch (amylose, amylopectin), cellulose, dextrin, and glucan (β1,3-glucan). Starch and cellulose are preferable.

Examples of the invert sugar include one obtained by hydrolyzing sucrose.

Examples of the compound having one carbon atom (C₁ compound) include methanol, formaldehyde, formate, carbon monoxide and carbon dioxide.

Examples of the lipid may include substances containing one or more saturated or unsaturated fatty acids of C₄ or more. In particular, an oil which is liquid at room temperature is preferable.

Examples of the oil may include plant oils such as soybean, corn, canola, Jatropha, palm, peanut, sunflower, coconut, mustard, cotton seed, palm kernel oil, olive, safflower, sesame, linseed, oily microbial cells, Chinese tallow tree, and animal oils.

Examples of the fatty acid may include compounds represented by a formula RCOOH (“R” represents a hydrocarbon group), which include unsaturated fatty acids and saturated fatty acids. Here, the unsaturated fatty acid is a compound having at least one double bond between two carbon atoms in “R”, and examples of the unsaturated fatty acid may include oleic acid, vaccenic acid, linoleic acid, palmitelaidic acid and arachidonic acid. The saturated fatty acid is a compound where the “R” is a saturated aliphatic group, and examples of the saturated fatty acid may include docosanoic acid, eicosanoic acid, octadecanoic acid, hexadecanoic acid, tetradecanoic acid, and dodecanoic acid. C₂ to C₂₂ fatty acids are preferable as the fatty acid, and C₁₂ fatty acid, C₁₄ fatty acid, C₁₆ fatty acid, C₁₈ fatty acid, C₂₀ fatty acid and C₂₂ fatty acid are more preferable.

The carbon source may also include salts and derivatives of these fatty acids and salts of these derivatives. Examples of the salt may include lithium salts, potassium salts and sodium salts.

Examples of the glycerine fatty acid ester include monoglyceride, diglyceride and triglyceride.

Examples of the polypeptide include microbial polypeptide and plant polypeptide. Here, examples of the microbial protein may include polypeptides obtainable from a yeast or bacterium, and examples of the plant protein may include polypeptides obtainable from soybean, corn, canola, Jatropha, palm, peanut, sunflower, coconut, mustard, cotton seed, palm kernel oil, olive, safflower, sesame and linseed.

Examples of the renewable carbon source may include biomass carbon sources. In particular, entirely or partially hydrolyzed biomass carbon sources are preferable. Examples of the biomass carbon source may include cellulose-based substrates such as waste materials of woods, papers and pulps, leafy plants, and fruit pulps; and partial plants such as stalks, grain particles, roots and tubers. Examples of the plants to be used as the biomass carbon source may include corn, wheat, rye, sorghum, triticale, rice, millet, barley, cassava, legumes such as peas, potato, sweet potato, banana, sugar cane, and tapioca. When the renewable carbon source such as biomass is added to the culture medium, the carbon source is preferably pretreated. Examples of the pretreatment may include an enzymatic pretreatment, a chemical pretreatment, and a combination thereof.

Examples of the carbon source may also include combinations of carbohydrate such as glucose with the lipid(s), the oil(s), the fats, the fatty acid(s) and glycerin fatty acid(s) ester(s).

Examples of the carbon source may also include the yeast extract and a combination of the yeast extract with the other carbon source such as glucose. The combination of the yeast extract with the C1 compound such as carbon dioxide and methanol is preferable.

For a nitrogen source, inorganic ammonium salts such as ammonium sulfate, ammonium chloride and ammonium phosphate, organic nitrogen such as hydrolyzed soybeans, ammonia gas, ammonia water, and the like can be used.

It is desirable to include required substances such as vitamin B1 and L-homoserine, or yeast extract and the like in an appropriate amount as an organic trace nutrient source.

For an inorganic matter (mineral), in addition thereto, potassium phosphate, magnesium sulfate, iron ion, manganese ion, and the like may be added in small amounts if necessary.

The culture medium used in the method according to the example is preferably a commercially available standard culture medium.

The standard culture medium is not particularly limited, and examples of the culture medium may include ready-made general media such as Luria Bertani (LB) broth. Sabouraud dextrose (SD) broth, and yeast medium (YM) broth. The medium suitable for the cultivation of the specific host can be selected appropriately for the use.

The isoprene produced with the cultured transformant is emitted as a gas from a fermentation tank. The isoprene gas is taken into a gas collecting apparatus, liquefied by cooling with a vapor trap, etc. equipped with a cooling apparatus, and thus can be recovered as liquid isoprene. At this time, the isoprene gas mainly passes through an adsorbent for adsorbing isoprene (e.g., hydrophobic silica gel and the like), and then its pressure is reduced as compared to the time of adsorption. Thereby, the gas with isoprene as a main component can be recovered (WO2014/156997A1). Moreover, it is preferable that the isoprene gas is subjected to condensation removal of moisture via cooling, in order to have moisture removed.

Examples of the isoprene recovery apparatus include the apparatus as described in the example described above.

The fermentation method is as described in the above.

—Polymerization Catalyst Composition—

An example of a polymerization catalyst composition used in the method for producing polyisoprene of the examples of this disclosure would be described in the following.

This polymerization catalyst composition necessarily contains:
a rare earth element compound (hereinafter referred to as “component (A)” as well); and
an organometallic compound (hereinafter referred to as “component (B)” as well).

Here, this polymerization catalyst composition may further contain:
an aluminoxane compound (hereinafter referred to as “component (C)” as well);
a halogen compound (hereinafter referred to as “component (D)” as well);
an ionic compound (hereinafter referred to as “component (E)” as well); and
a compound capable of serving as an anionic ligand (hereinafter referred to as “component (F)” as well).

—Rare Earth Element Compound (Component (A))—

The component (A) may be a rare earth element-containing compound or a reaction product of the rare earth element-containing compound and a Lewis base, which has metal-nitrogen bond (M-N bond).

Note that examples of the rare earth containing compound include to a compound containing scandium, yttrium, or lanthanoid composed of elements with atomic numbers from 57 to 71. Specific examples of the lanthanoid element include lanthanum, cerium, praseodymium, neodymium, promethium, samarium, europium, gadolinium, terbium, dysprosium, holmium, erbium, thulium, ytterbium, and lutetium.

Examples of the Lewis base include tetrahydrofuran, diethyl ether, dimethylaniline, trimethylphosphine, lithium chloride, neutral olefins, and neutral diolefins.

Here, it is preferable that the rare earth element-containing compound or the reaction product of the rare earth element-containing compound and the Lewis base has no bond between the rare earth element and carbon. When the reaction product of the rare earth element-containing compound and the Lewis base has no bond between the rare earth element and carbon, an obtained compound is stable and easy to handle.

Note that the component (A) may be used alone or in a combination of two or more.

Here, the component (A) is preferably a compound represented with the formula (S1):

M-(NQ¹)(NQ²)(NQ³) (S1)

where M is at least one element selected from scandium, yttrium or lanthanide elements; NQ¹, NQ², and NQ³ are amide groups and may be either the same or different from one another; and the compound has three M-N bonds.

According to the aforementioned configuration, it is possible to use a compound having 3 M-N bonds as the component (A), in which each bond is chemically equivalent, rendering the structure of the compound stable and easy to handle. Moreover, according to the aforementioned configuration, the catalytic activity in the reaction system may be further improved. Therefore, the reaction time may be further shortened, and the reaction temperature may be further raised.

The amide groups represented by NQ¹, NQ² and NQ³ may be any one of an aliphatic amide group such as a dimethylamide group, a diethylamide group, and a diisopropyl amide group; a phenylamide group, a 2,6-di-tert-butylphenyl amide group, a 2,6-diisopropylphenyl amide group, a 2,6-dineopentilphenyl amide group, a 2-tert-butyl-6-isopropyphenyl amide group, a 2-tert-butyl-6-neopentilphenyl amide group, and a 2-isopropyl-6-neopentilphenyl amide group; an arylamide group such as a 2,4,6-tert-butylphenyl amide group; and a bis-trialkyl silylamide group such as a bis-trimethyl silylamide group. Among them, the bis-trimethylsilyl amide group is preferable.

Such amide groups may be used alone or in a combination of two or more.

—Organometallic Compound (Component (B))—

The component (B) is a compound represented with the formula (S2):

YR⁴ₐR⁵_{b}R⁶_{c} (S2)

where Y is a metallic element selected from the group consisting of Group 1, Group 2, Group 12 and Group 13; R⁴ and R⁵ are C1 to C10 hydrocarbon group or hydrogen atom, and may be either identical or different; R⁶ is a C1 to C10 hydrocarbon group; R⁶ may be either identical to or different from the R⁴ or R⁵; if Y is a Group 1 metallic element, a is 1, and b, c are 0; if Y is a Group 2 metallic element or a Group 12 metallic element, a and b are 1, and c is 0; and if Y is a Group 13 metallic element, a, b and c are 1.

The component (B) is preferably an organoaluminum compound represented with the formula (S3):

AlR⁷R⁸R⁹ (S3)

where R⁷ and R⁸ are C1 to C10 hydrocarbon group or hydrogen atom, and may be either identical or different; R⁹ is a C1 to C10 hydrocarbon group; and R⁹ may be either identical to or different from the R⁷ or R⁸.

As the organoaluminum compound in general formula (X), trimethyl aluminum, triethyl aluminum, tri-n-propyl aluminum, triisopropyl aluminum, tri-n-butyl aluminum, triisobutyl aluminum, tri-t-butyl aluminum, tripentyl aluminum, trihexyl aluminum, tricyclohexyl aluminum, trioctyl aluminum; diethylaluminum hydride, di-n-propyl aluminum hydride, di-n-butyl aluminum hydride, diisobutyl aluminum hydride, dihexyl aluminum hydride, diisohexyl aluminum hydride, dioctyl aluminum hydride, diisooctyl aluminum hydride; ethyl aluminum dihydride, n-propyl aluminum dihydride, isobutyl aluminum dihydride may be used, among which triethyl aluminum, triisobutyl aluminum, diethylaluminum hydride, diisobutyl aluminum hydride are preferable.

Such organoaluminum compounds may be used alone or in a combination of two or more.

—Aluminoxane Compound (Compound (C))—

The compound (C) is a compound obtainable by bringing an organoaluminum compound into contact with a condensing agent.

By using the component (C), the catalytic activity in the polymerization reaction system may be further improved. Therefore, the reaction time may be further shortened, and the reaction temperature may be further raised.

Here, examples of the organoaluminum compound include trialkyl aluminum such as trimethyl aluminum, triethyl aluminum, triisobutyl aluminum, etc., and a mixture thereof. Among them, a mixture of trimethyl aluminum and tributyl aluminum is preferable.

Examples of the condensing agent include one ordinarily used in the art.

Examples of the component (C) include an aluminoxane represented with the formula (S4):

—(Al(R¹⁰)O)ₙ— (S4)

where R¹⁰ represents a C1 to C10 hydrocarbon group, and may be one that is partially substituted for by a halogen atom and/or an alkoxy group; R¹⁰ may be either identical or different among repeating units; and n is 5 or more.

The molecular structure of the aforementioned aluminoxane may be either straight chain or cyclic. Moreover, n is preferably 10 or more. Further, examples of the hydrocarbon group of R¹⁰ include methyl group, ethyl group, propyl group, isobutyl group, etc. In particular, methyl group is preferable. Such hydrocarbon groups may be used alone or in a combination of two or more. The hydrocarbon group of R¹⁰ is preferably a combination of methyl group and isobutyl group.

Examples of the component (C) include TMAO, e.g., trade name: TMAO-341, manufactured by Tosoh Finechem Corporation.

Moreover, examples of the component (C) include MMAO, e.g., trade name: MMAO-3A, manufactured by Tosoh Finechem Corporation.

Further, examples of the component (C) include PMAO, e.g., trade name: TMAO-211, manufactured by Tosoh Finechem Corporation.

Among these, from the viewpoint of improving the effect of catalytic activity enhancement, TMAO and MMAO are preferable, and from the viewpoint of further improving the effect of catalytic activity enhancement, TMAO is more preferable.

—Halogen Compound (Component (D))—

The component (D) is at least one compound selected from the group consisting of: a halogen containing compound, which is a Lewis acid (hereinafter referred to as “component (D-1)” as well), a complex compound of metal halides and a Lewis base (hereinafter referred to as “component (D-2)” as well), and an organic compound containing an active halogen (hereinafter referred to as “component (D-3)” as well).

These compounds react with the component (A), i.e., a rare earth element-containing compound or a reaction product of the rare earth element-containing compound and a Lewis base, which has M-N bond, thereby generating a cationic transition metal compound, a halogenated transition metal compound, and/or a transition metal compound in a state where a transition metal center has insufficient electrons.

By using the component (D), the cis-1,4-bond content of polyisoprene may be raised.

Examples of the component (D-1) include a halogen containing compound containing an element of Group 3, Group 4, Group 5, Group 6, Group 8, Group 13, Group 14 or Group 15. In particular, an aluminum halide or an organometallic halide is preferable.

Examples of the halogen containing compound, which is a Lewis acid, include titanium tetrachloride, tungsten hexachloride, tri(pentafluorophenyl) borate, methyl aluminum dibromide, methyl aluminum dichloride, ethyl aluminum dibromide, ethyl aluminum dichloride, butyl aluminum dibromide, butyl aluminum dichloride, dimethyl aluminum bromide, dimethyl aluminum chloride, diethyl aluminum bromide, diethyl aluminum chloride, dibutyl aluminum bromide, dibutyl aluminum chloride, methyl aluminum sesquibromide, methyl aluminum sesquichloride, ethyl aluminum sesquibromide, ethyl aluminum sesquichloride, aluminum tribromide, tri(pentafluorophenyl aluminum, dibutyltin dichloride, tin tetrachloride, phosphorus trichloride, phosphorus pentachloride, antimony trichloride and antimony pentachloride. In particular, ethyl aluminum dichloride, ethyl aluminum dibromide, diethyl aluminum chloride, diethyl aluminum bromide, ethyl aluminum sesquichloride and ethyl aluminum sesquibromide are preferable.

As the halogen, chlorine or bromine is preferable.

The halogen containing compound, which is a Lewis acid, may be used alone or in a combination of two or more.

Examples of the metal halide used in the component (D-2) include beryllium chloride, beryllium bromide, beryllium iodide, magnesium chloride, magnesium bromide, magnesium iodide, calcium chloride, calcium bromide, calcium iodide, barium chloride, barium bromide, barium iodide, zinc chloride, zinc bromide, zinc iodide, cadmium chloride, cadmium bromide, cadmium iodide, mercury chloride, mercury bromide, mercury iodide, manganese chloride, manganese bromide, manganese iodide, rhenium chloride, rhenium bromide, rhenium iodide, copper chloride, copper iodide, silver chloride, silver bromide, silver iodide, gold chloride, gold iodide, gold bromide, etc. In particular, magnesium chloride, calcium chloride, barium chloride, zinc chloride, manganese chloride and copper chloride are preferable, and magnesium chloride, zinc chloride, manganese chloride and copper chloride are more preferable.

The Lewis base used in the component (D-2) is preferably a phosphorus compound, a carbonyl compound, a nitrogen compound, an ether compound, or an alcohol.

For example, tributyl phosphate, tri-2-ethylhexyl phosphate, triphenyl phosphate, tricresyl phosphate, triethylphosphine, tributylphosphine, triphenylphosphine, diethylphosphino ethane, diphenylphosphino ethane, acetylacetone, benzoylacetone, propionitrileacetone, valerylacetone, ethyl acetylacetone, methyl acetoacetate, ethyl acetoacetate, phenyl acetoacetate, dimethyl malonate, diethyl malonate, diphenyl malonate, acetic acid, octanoic acid, 2-ethylhexanoic acid, oleic acid, stearic acid, benzoic acid, naphthenic acid, versatic acid (a trade name manufactured by Shell Chemicals, which is a synthetic acid containing a mixture of isomers of C₁₀ monocarboxylic acid), triethylamine, N,N-dimethylacetamide, tetrahydrofuran, diphenyl ether, 2-ethylhexyl alcohol, oleyl alcohol, stearyl alcohol, phenol, benzyl alcohol, 1-decanol, lauryl alcohol and the like may be mentioned. In particular, tri-2-ethylhexyl phosphate, tricresyl phosphate, acetylacetone, 2-ethylhexane acid, versatic acid, 2-ethylhexyl alcohol, 1-decanol, and lauryl alcohol are preferable.

The Lewis base mentioned above is brought to reaction at a ratio of 0.01 to 30 mol, preferably 0.5 to 10 mol per mole of the metal halide mentioned above. A reaction product with the Lewis base at this ratio allows a reduction in metal remaining in the polymer.

Examples of the component (D-3) include benzyl chloride and the like.

—Ionic Compound (Component (E))—

As the component (E) as described above may be an ionic compound and the like that includes non-coordinating anion and cation.

Examples of the non-coordinating anion include tetravalent boron anion, such as tetraphenyl borate, tetrakis(monofluorophenyl)borate, tetrakis(difluorophenyl)borate, tetrakis(trifluorophenyl)borate, tetrakis(tetrafluorophenyl)borate, tetrakis(pentafluorophenyl)borate, tetrakis(tetrafluoromethylphenyl)borate, tetra(tolyl) borate, tetra(xylyl)borate, (triphenyl,pentafluorophenyl)borate, [tris(pentafluorophenyl)phenyl]borate, tridecahydride-7,8-dicarbaundecaborate, etc.

Such non-coordinating anion may be used alone or in a combination of two or more.

Examples of the cation include carbonium cation, oxonium cation, ammonium cation, phosphonium cation, cycloheptatrienyl cation, and ferrocenium cation having transition metal. Here, examples of the carbonium cation include trisubstituted carbonium cation such as triphenyl carbonium cation, tri(substituted phenyl) carbonium cation and the like. Moreover, examples of the tri(substituted phenyl) carbonium cation include tri(methylphenyl) carbonium cation, tri(dimethylphenyl) carbonium cation and the like.

Examples of the ammonium cation include trialkyl ammonium cation such as trimethyl ammonium cation, triethyl ammonium cation, tripropyl ammonium cation, and tributyl ammonium cation; N,N-dialkyl anilinium cation such as N,N-dimethyl anilinium cation, N,N-diethyl anilinium cation, N,N-2,4,6-pentamethyl anilinium cation and the like; and dialkyl ammonium cation such as diisopropyl ammonium cation, dicyclohexyl ammonium cation and the like.

Examples of the phosphonium cation include triarylphosphonium cation such as triphenylphosphonium cation, tri(methylphenyl)phosphonium cation, tri(dimethylphenyl)phosphonium cation and the like.

Such cation may be used alone or in a combination of two or more.

In particular, preferred examples include N,N-dimethylanilinium tetrakis(pentafluorophenyl)borate and triphenylcarbonium tetrakis(pentafluorophenyl)borate.

—Compound Capable of Serving as an Anionic Ligand—

Examples of the component (F) include an anionic tridentate ligand precursor represented with the formula (S5) (see Organometallics, 23, p 47784787 (2004)).

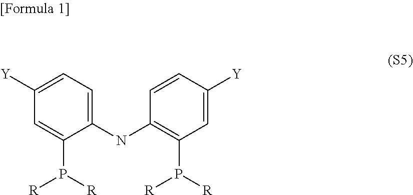

(in the formula, R represents an alkyl group or an aryl group, Y represents at least one selected from the group consisting of a hydrogen, an alkyl group, a halogen group, and a silyl group)

In particular, a PNP ligand such as bis(2-diphenylphosphinophenyl) amine and the like may be mentioned.

According to the method for producing polyisoprene of this disclosure, it is possible to obtain the effect of the isoprene-containing composition of this disclosure, i.e., the capability of producing a polyisoprene with a comparatively high molecular weight.

A weight ratio of each component in the polymerization catalyst composition used in the method for producing polyisoprene of the examples of this disclosure would be described in the following.

From the viewpoint of improving the catalytic activity in the reaction, a molar ratio of the component (B) (the organometallic compound) to the component (A) is preferably 1 to 50.

From the viewpoint of improving the catalytic activity in the reaction, a molar ratio of the aluminum in the component (C) to the rare earth element in the component (A) is preferably 10 to 1000.

From the viewpoint of improving the catalytic activity in the reaction, a molar ratio of the component (D) (the halogen compound) to the component (A) is preferably 0.1 to 50.

From the viewpoint of improving the catalytic activity in the reaction, a molar ratio of the component (E) (the ionic compound) to the component (A) is preferably 0.1 to 10.

Here, in the method for producing polyisoprene of the examples of this disclosure, from the viewpoint of sufficiently activating the polymerization reaction, a compounding amount of the rare earth element compound is preferably 0.018 parts by mass or more, more preferably 0.02 parts by mass or more, preferably 0.55 parts by mass or more, more preferably 0.6 parts by mass or more, further preferably 0.65 parts by mass or more, particularly preferably 0.7 parts by mass or more per 100 parts by mass of the isoprene-containing composition. Moreover, from the viewpoint of reducing the risk of deactivating the polymerization reaction in contrary, the compounding amount is preferably 0.95 parts by mass or less, more preferably 0.9 parts by mass or less, further preferably 0.85 parts by mass or less, particularly preferably 0.8 parts by mass or less per 100 parts by mass of isoprene-containing composition.

Here, in the method for producing polyisoprene of the examples of this disclosure, from the viewpoint of sufficiently activating the polymerization reaction, a compounding amount of the organometallic compound is necessarily 0.6 parts by mass or more, preferably 0.9 parts by mass or more, more preferably 1.1 parts by mass or more per 100 parts by mass of the isoprene-containing composition.

Moreover, from the viewpoint of reducing the risk of deactivating the polymerization reaction in contrary, the compounding amount of the organometallic compound is necessarily 3.0 parts by mass or less, preferably 1.6 parts by mass or less, more preferably 1.5 parts by mass or less or less per 100 parts by mass of the isoprene-containing composition.

(Polyisoprene)

The polyisoprene (hereinafter referred to as “the polyisoprene” as well) produced with the method for producing polyisoprene of the examples of this disclosure would be described in the following.

The number-average molecular weight (Mn) of the polyisoprene is preferably 1,000,000 or more, more preferably 1,300,000 or more.

The molecular weight distribution (Mw/Mn) thereof is preferably 2.5 or less, more preferably 2.0 or less.

Note that the number-average molecular weight (Mn) and the molecular weight distribution (Mw/Mn) may be measured with the method as described in the examples mentioned below.

The cis-1,4-bond content of the polyisoprene is preferably 98% or more, more preferably 99% or more, without being limited thereto. A high value may enhance an elongation-induced crystallization ability of the polyisoprene, and enhance an elasticity of the polyisoprene.

A trans-1,4-bond content of the polyisoprene is preferably 2.0% or less, more preferably 1.0% or less, without being limited thereto. A lower value may enhance the elongation-induced crystallization ability of the polyisoprene, and enhance the elasticity of the polyisoprene.

A 1,2-vinyl bond content of the polyisoprene is preferably 2.0% or less, more preferably 1.0% or less, without being limited thereto. A lower value may enhance the elongation-induced crystallization ability of the polyisoprene, and enhance the elasticity of the polyisoprene.

A 3,4-vinyl bond content of the polyisoprene is preferably 2.0% or less, more preferably 1.0% or less, without being limited thereto. A lower value may enhance the elongation-induced crystallization ability of the polyisoprene, and enhance the elasticity of the polyisoprene.

(Rubber Composition)

The rubber composition containing the polyisoprene (hereinafter referred to as “the rubber composition” as well) would be described in the following. Here, the polyisoprene of this disclosure may be a rubber component in the rubber composition.

The rubber composition may contain rubber components other than the polyisoprene, and may further contain a filler, an age resistor, a softener, a stearic acid, a zinc oxide, a vulcanization accelerator, a vulcanizing agent, an oil, a sulfur, etc.

The rubber composition may be produced with a method well-known to a skilled person.

(Tire, Rubber Product)

A tire may be produced by using the rubber composition. Here, any member of the tire may be produced by using the rubber composition. The tire may be produced with a method well-known to a skilled person.

Moreover, rubber products other than tire, such as footwear, belt, flooring, etc., may be produced by using the rubber composition as well.

### EXAMPLES

The present disclosure is described in more detail below with reference to Examples, by which the present disclosure is not intended to be limited in any way.

Note that the following examples use Pantoea ananatis as the host cell, one derived from Mucuna as the isoprene synthase, one derived from Saccharomyces cerevisiae, Enterococcus faecalis or Metanocella pardicola as the enzyme involved in the mevalonic acid pathway. Here, the isoprene synthase is transduced into the host cell via chromosome fixation of the enzyme involved in the mevalonic acid pathway by using a expression plasmid.

### Example 1

(1) Preparation of Isoprene-Containing Composition (Preparation of Fermented Isoprene)

(1-1) Preparation of Plasmid for Upstream of MVA Pathway for Chromosome Fixation

(1-1-1) Construction of Arabinose-Inducible Plasmid for Expressing Mevalonate Pathway Upstream Gene (mvaES [Enterococcus faecalis (E. faecalis)]) Derived from E. faecalis, pMW-Pₐᵣₐ-mvaES-Tₜᵣₚ

(1-1-1-1) Chemical Synthesis and Cloning of mvaES (E. faecalis) Gene

A nucleotide sequence (GenBank/EMBL/DDBJ accession ID AF290092.1) and an amino acid sequence (mvaS, GenPept accession ID AAG02438.1, mvaE, GenPept accession ID AAG02439.1) of the mvaES gene encoding the mevalonate pathway upstream gene (mvaES) (Enterococcus faecalis) (E. faecalis) derived from E. faecalis are known publicly (see Wilding, E I et al., J. Bacteriol. 182 (15), 4319-4327 (2000), which is incorporated herein by reference in its entirety). Based on this information, a mvaE gene and a mvaS gene in which their codon usages had been optimized for E. coli were designed and designated as EFmvaE and EF mvaS, respectively. Nucleotide sequences of EFmvaE and EFmvaS are shown in SEQ ID NO:1 and SEQ ID NO:2, respectively. DNA sequences of EFmvaE and EFmvaS prepared by chemical synthesis were cloned into a plasmid for expression pUC57 (supplied from GenScript), and designated as pUC57-EFmvaE and pUC57-EFmvaS, respectively. Nucleotide sequences of pUC57-EFmvaE and pUC57-EFmvaS are shown in SEQ ID NO:3 and SEQ ID NO:4, respectively.

(1-1-1-2) Preparation for Construction of Plasmid pMW-P_{trc}-mvaES-Tₜᵣₚ Used for In-Fusion Method

A plasmid pMW-P_{trc}-mvaES-Tₜᵣₚ used for the In-fusion method was constructed according to the following procedure.

A plasmid pMW219 (supplied from Nippon Gene, Part number: 310-02571) was digested with SmaI, and this digested plasmid was purified. The resulting plasmid was designated as pMW219/SmaI.

In order to obtain a gene of a trc promoter (P_{trc}) region, PCR was carried out using a plasmid pTrcHis2B having the P_{trc} region as the template and using synthesized oligonucleotides consisting of the nucleotide sequences of SEQ ID NO:5 and SEQ ID NO:6 as primers.

In order to obtain a mvaE gene portion, PCR was carried out using the plasmid pUC57-EFmvaE as the template and using synthesized oligonucleotides consisting of the nucleotide sequences of SEQ ID NO:7 and SEQ ID NO:8 as the primers.

In order to obtain a mvaS gene portion, PCR was carried out using the plasmid pUC57-EFmvaS as the template and using synthesized oligonucleotides consisting of the nucleotide sequences of SEQ ID NO:8 and SEQ ID NO:9 as the primers.

In order to obtain a gene of a trp terminator (Tₜᵣₚ) region, PCR was carried out using a plasmid pSTV-P_{tac}-Tₜᵣₚ having the Tₜᵣₚ region as the template and using synthesized oligonucleotides consisting of the nucleotide sequences of SEQ ID NO:10 and SEQ ID NO:11 as the primers.

In the above four PCR cases, PrimeStar polymerase (supplied from TaKaRa Bio) was used as the enzyme, a reaction solution was prepared according to instructions provided by the supplier of the enzyme, and the reaction of a cycle of 98° C. for 10 seconds, 55° C. for 5 seconds and 72° C. for 60 seconds/kb was repeated 30 times. As a result, the PCR product comprising the gene of the P_{trc} region, the mvaE gene, the mvaS gene and the gene of the Tₜᵣₚ region was obtained.

(1-1-1-3) Construction of Plasmid pMW-P_{trc}-mvaES-Tₜᵣₚ Used for In-Fusion Method

Subsequently, PCR was carried out using the purified PCR product comprising P_{trc} and the purified PCR product comprising the mvaE gene as the template and using synthesized oligonucleotides consisting of SEQ ID NO:5 and SEQ ID NO:8 as the primers. Also PCR was carried out using the purified PCR product comprising the mvaS gene and the purified PCR product comprising Tₜᵣₚ as the template and using synthesized oligonucleotides consisting of SEQ ID NO:9 and SEQ ID NO:12 as the primers.

As a result, the PCR product comprising the gene of the P_{trc} region and the mvaE gene, and the PCR product comprising the mvaS gene and the gene of the Tₜᵣₚ region were obtained.

Subsequently, the PCR product comprising the gene of the P_{trc} region and the mvaE gene, the PCR product comprising the mvaS gene and the gene of the Tₜᵣₚ region, and the plasmid pMW219/SmaI digested above were ligated using In-Fusion HD Cloning Kit (supplied from Clontech). The resulting plasmid was designated as pMW-P_{trc}-mvaES-Tₜᵣₚ. A sequence of obtained pMW-P_{trc}-mvaES-Tₜᵣₚ, is shown in SEQ ID NO:13.

(1-1-1-4) Construction of Plasmid pMW-Pₐᵣₐ-mvaES-Tₜᵣₚ

The arabinose-inducible plasmid for expression of a mevalonate pathway upstream gene, pMW-Pₐᵣₐ-mvaES-Tₜᵣₚ was constructed according to the following procedure.

PCR was carried out using the plasmid pMW-P_{trc}-mvaES-Tₜᵣₚ prepared in (1-1-3) as the template and using synthesized oligonucleotides consisting of the nucleotide sequences of SEQ ID NO:14 and SEQ ID NO:15 as the primers.

PCR was carried out using a plasmid pKD46 (see Proc. Natl. Acad. Sci. USA, 2000, vol. 97, No. 12, p 6640-6645, which is incorporated herein by reference in its entirety) comprising a gene of a P_{araC} region, an araC gene and a gene of a P_{araBAD} region (hereinafter also collectively referred to as a “gene of a Pₐᵣₐ region”) as the template and using synthesized oligonucleotides consisting of the nucleotide sequences of SEQ ID NO:16 and SEQ ID NO:17 as the primers.

As a result, the PCR product comprising the plasmid pMW and the mvaES gene and the PCR product comprising the gene of the Pₐᵣₐ region were obtained. Purified these PCR products were ligated using In-Fusion HD Cloning Kit (supplied from Clontech). The resulting arabinose-inducible plasmid for expression of the mevalonate pathway upstream gene derived from E. faecalis (mvaES (E. faecalis)) was designated as pMW-Pₐᵣₐ-mvaES-Tₜᵣₚ. A nucleotide sequence of pMW-Pₐᵣₐ-mvaES-Tₜᵣₚ is shown in SEQ ID NO:18.

(1-1-2) Construction of Integrative Conditional Replication Plasmid Possessing Mevalonate Pathway Upstream

A vector pAH162-λattL-Tc^{R}-λattR (Minaeva N I et al., BMC Biotechnol. 2008; 8:63, which is incorporated herein by reference in its entirety) was used in order to construct an integrative plasmid possessing an upstream gene and a downstream gene of the mevalonate pathway.

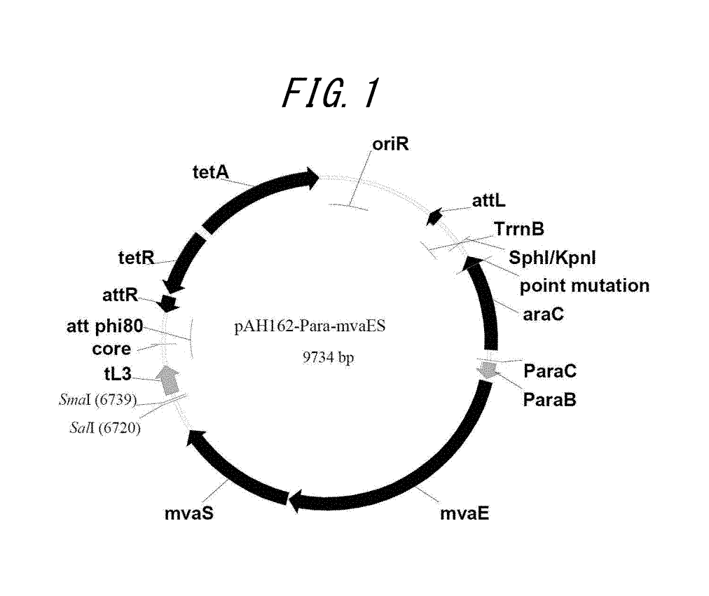

A KpnI-SalI fragment of pMW-Pₐᵣₐ-mvaES-Tₜᵣₚ was cloned into a recognition site for SphI-SalI in pAH162-λattL-Tc^{R}-λattR. As a result, a plasmid pAH162-Pₐᵣₐ-mvaES possessing an operon mvaES derived from E. faecalis under the control of the Para promoter and a repressor gene araC from E. coli was constructed (FIG. 1).

(1-2) Preparation of Plasmid for Downstream of MVA Pathway for Chromosome Fixation

(1-2-1) Construction of Integrative Plasmid pAH162-Km-Ptac-KDyI

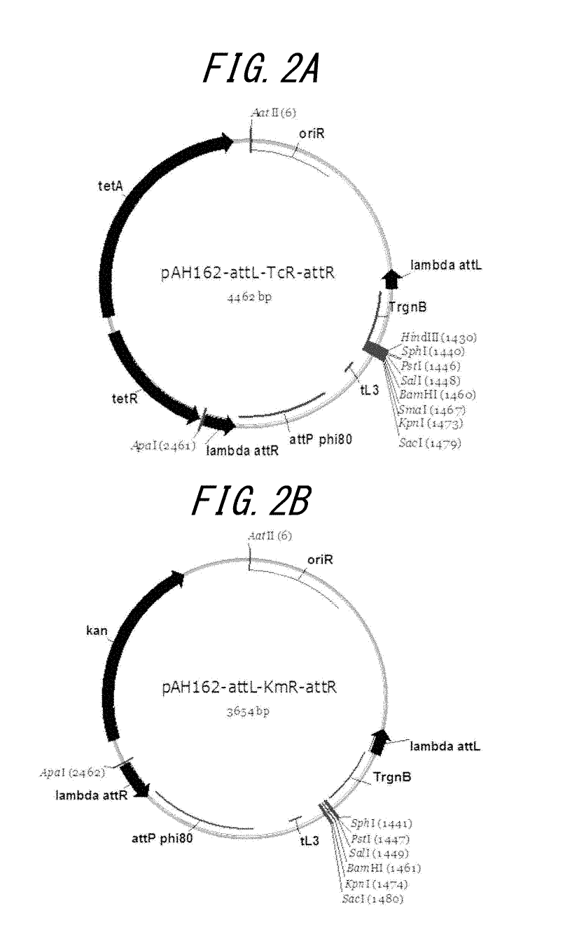

An AatII-ApaI fragment of an integrative plasmid pAH162-λattL-Tc^{R}-λattR (Minaeva N I et al. BMC Biotechnol. 2008; 8:63, which is incorporated herein by reference in its entirety) comprising a tetAR gene (FIG. 2A) was substituted with a DNA fragment obtained by PCR using synthesized oligonucleotides consisting of the nucleotide sequences of SEQ ID NO:19 (primer 11) and SEQ ID NO:20 (primer 12) as the primers and using a plasmid pUC4K (Taylor L A and Rose R E. Nucleic Acids Res. 16, 358, 1988, which is incorporated herein by reference in its entirety) as the template. As a result, pAH162-λattL-Km^{R}-λattR was obtained (FIG. 2B).

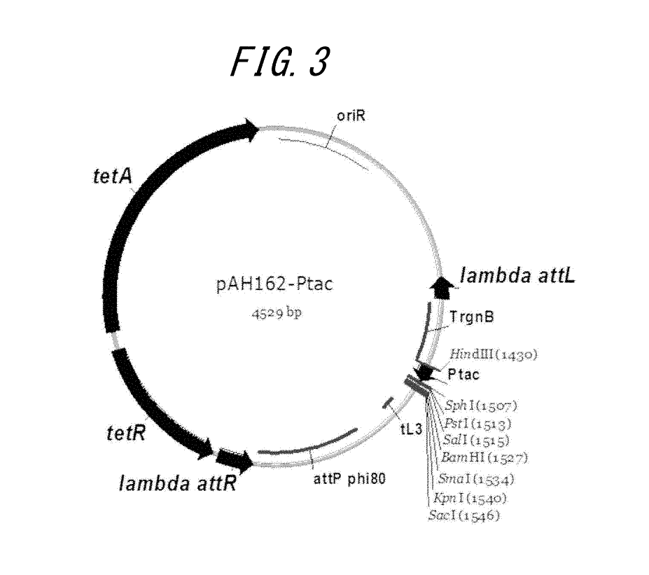

The Ptac promoter was inserted into a site HindIII-SphI in the integrative vector pAH162-λattL-Tc^{R}-λattR. As a result, an integrative vector pAH162-P_{tac} was constructed. The cloned promoter fragment was sequenced. A map of pAH162-P_{tac} is shown in FIG. 3.

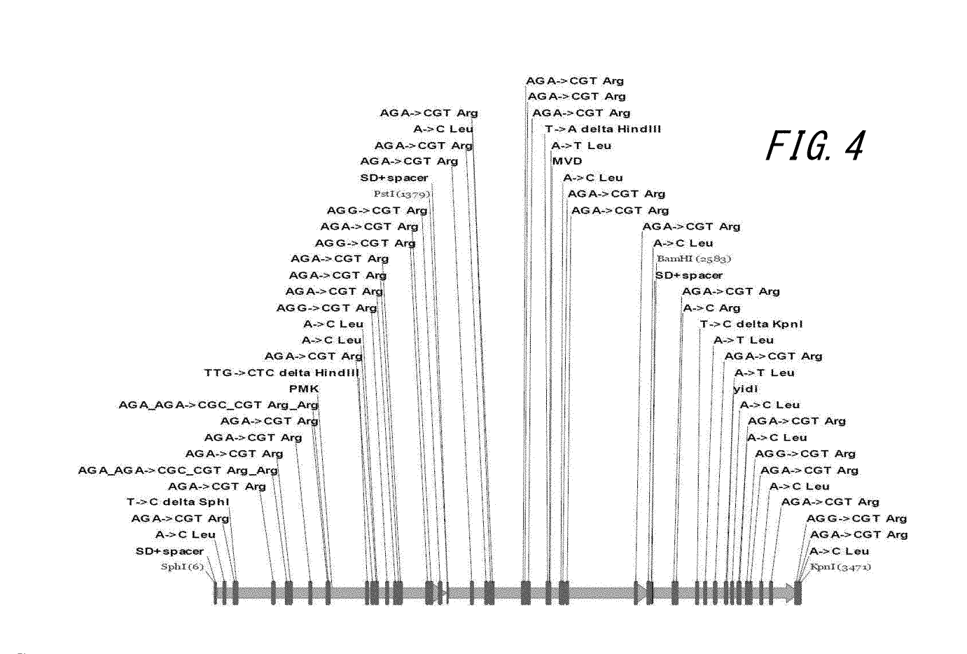

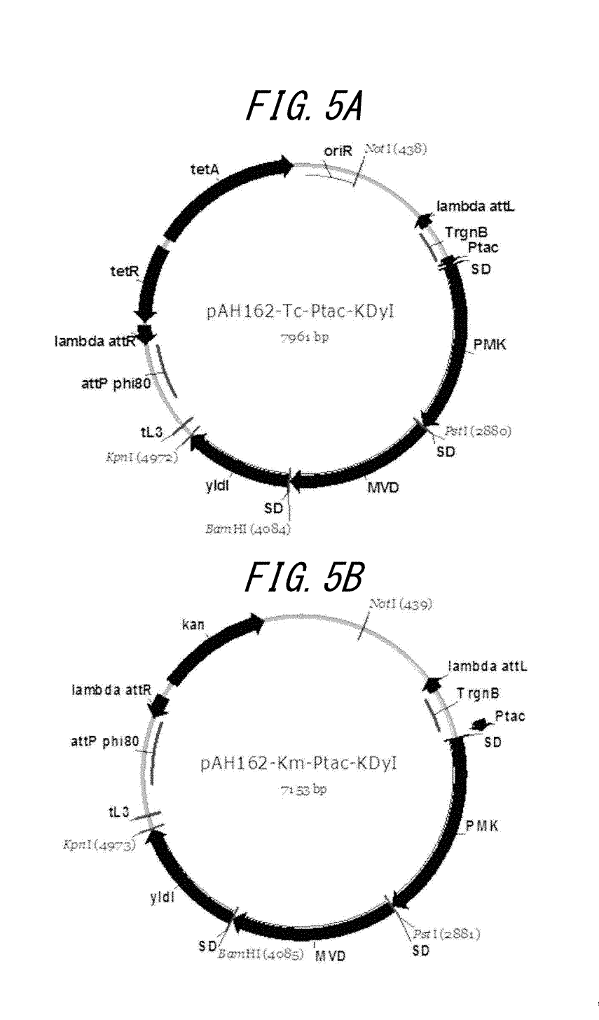

Regarding a DNA fragment holding the PMK gene, the MVD gene and the yIDI gene derived from S. cerevisiae (KDyIoperon), a codon was optimized by substituting rare codons, and the chemically synthesized DNA fragment with the codon optimized was obtained from ATG Service Gene (Russia) (SEQ ID NO:21) (FIG. 4), and was subcloned into a site SphI-KpnI in the integrative vector pAH162-P_{tac}. The resulting plasmid pAH162-Tc-P_{tac}-KDyI holding an expression cassette P_{tac}-KDyI is shown in FIG. 5A. Subsequently, a NotI-KpnI fragment of pAH162-Tc-P_{tac}-KDyI holding a tetAR gene was substituted with a corresponding fragment of pAH162-λattL-Km^{R}-λattR. As a result, a plasmid pAH162-Km-P_{tac}-KDyI having a kanamycin resistant gene kan as a marker was obtained (FIG. 5B).

(1-2-2) Construction of pAH162-P_{tac}-mvk (M. paludicola) Intergrative Plasmid

A chemically synthesized DNA fragment (SEQ ID NO:22) comprising a coding portion of a presumed mevalonate kinase gene (mvk gene) derived from SANAE (for complete genome sequence, see GenBank Accession Number AP011532) that was Methanocella paludicola and ligated to a standard SD sequence was cloned into a site PstI-KpnI in the above integrative expression vector pAH162-Ptac, to thereby obtain a pAH162-P_{tac}-mvk (M. paludicola) plasmid.

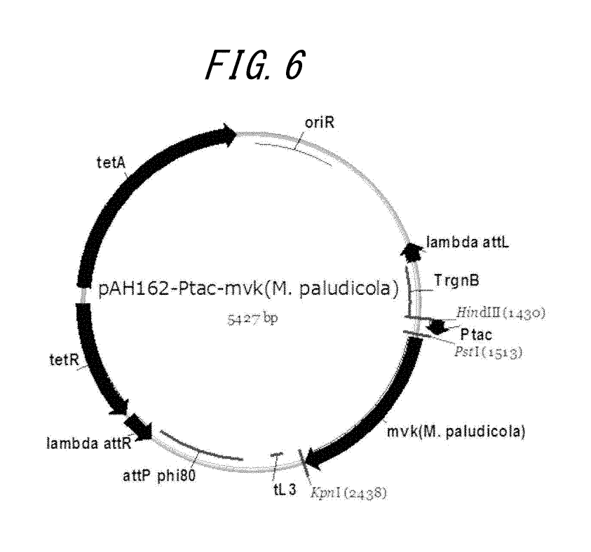

A map of the integrative plasmid pAH162-P_{tac}-mvk holding the mvk gene is shown in FIG. 6.

(1-3) Construction of Isoprene-Producing Bacterium

(1-3-1) Construction of Recipient Strain SC17(0) ΔampC::attBₚₕᵢ₈₀ ΔampH::attBₚₕᵢ₈₀ Δcrt::P_{tac}-mvk (M. paludicola)

Chromosomal modifications ΔampH::attBₚₕᵢ₈₀ and ΔampC::attBₚₕᵢ₈₀ were introduced into P. ananatis SC17(0) stepwise using two step technique comprising λRed dependent integration of a PCR-amplified DNA fragment comprising the gene kan flanking to attLₚₕᵢ₈₀ and attRₚₕᵢ₈₀ and a 40 bp sequence homologous to a target chromosome site (Katashkina J I et al. BMC Mol Biol. 2009; 10:34, which is incorporated herein by reference in its entirety), followed by removal according to the previously reported technique of the kanamycin resistant marker (Andreeva I G et al. FEMS Microbiol Lett. 2011; 318(1):55-60, which is incorporated herein by reference in its entirety). SC17(0) is a λRed resistant derivative of P. ananatis AJ13355 (Katashkina J I et al. BMC Mol Biol. 2009; 10:34, which is incorporated herein by reference in its entirety); an annotated complete genome sequence of P. ananatis AJ13355 is available as PRJDA162073 or GenBank Accession Numbers AP012032.1 and AP012033.1. DNA fragments each used for the integration into ampH and ampC genes, respectively were formed by PCR using a plasmid pMWattphi (Minaeva N I et al. BMC Biotechnol. 2008; 8:63, which is incorporated herein by reference in its entirety) as the template and using synthesized oligonucleotides consisting of the nucleotide sequences of SEQ ID NO:23 (primer 13) and SEQ ID NO:24 (primer 14) and synthesized oligonucleotides consisting of the nucleotide sequences of SEQ ID NO:25 (primer 15) and SEQ ID NO:26 (primer 16) as the primers. The resulting chromosomal modification was verified by PCR using synthesized oligonucleotides consisting of the nucleotide sequences of SEQ ID NO:27 (primer 17) and SEQ ID NO:28 (primer 18) and synthesized oligonucleotides consisting of the nucleotide sequences of SEQ ID NO:29 (primer 19) and SEQ ID NO:30 (primer 20) as the primers.

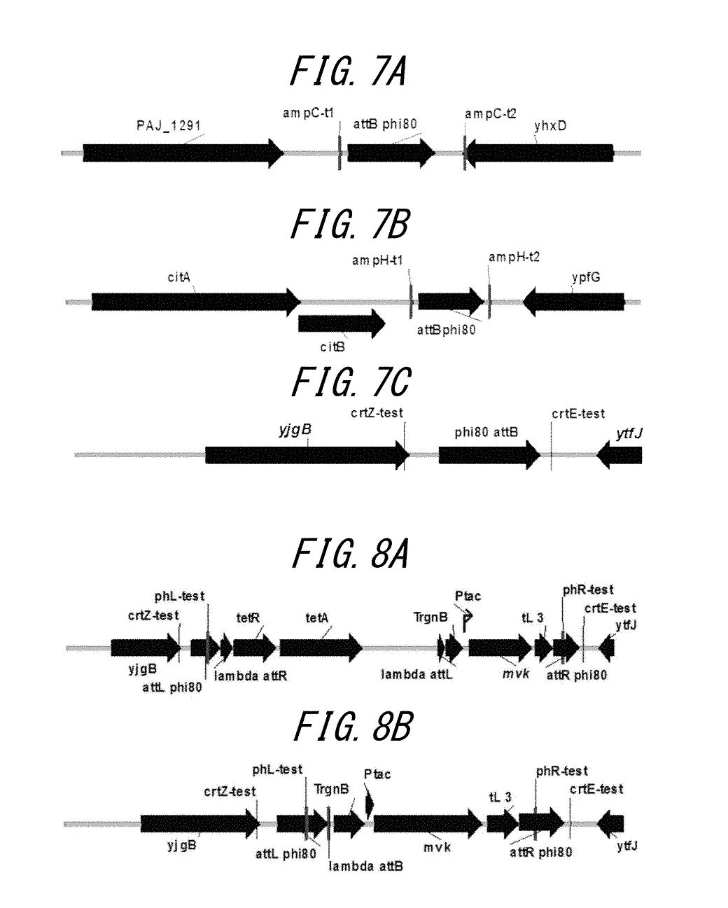

In parallel, a derivative of P. ananatis SC17(0) holding an attB site of the phage phi80 in place of an operon crt (positioned on a megaplasmid pEA320 (320 kb) that is a portion of the genome of P. ananatis AJ13355) was constructed. In order to obtain this strain, the λRed dependent integration of a PCR-amplified DNA fragment holding attLₚₕᵢ₈₀-kan-attRₚₕᵢ₈₀ flanking to a 40 bp region homologous to a target site in the genome was carried out according to the previously described technique (Katashkina J I et al. BMC Mol Biol. 2009; 10:34, which is incorporated herein by reference in its entirety). A DNA fragment used for substitution of the operon crt with attLₚₕᵢ₈₀-kan-attRₚₕᵢ₈₀ was amplified by PCR using synthesized oligonucleotides consisting of the nucleotide sequences of SEQ ID NO:31 (primer 21) and SEQ ID NO:32 (primer 22). The plasmid pMWattphi (Minaeva N I et al. BMC Biotechnol. 2008; 8:63, which is incorporated herein by reference in its entirety) was used as the template in this reaction. The resulting integrant was designated as SC17(0)Δcrt::attLₚₕᵢ₈₀-kan-attRₚₕᵢ₈₀. A chromosomal structure of SC17(0)Δcrt::attLₚₕᵢ₈₀-kan-attRₚₕᵢ₈₀ was verified by PCR using synthesized oligonucleotides consisting of the nucleotide sequences of SEQ ID NO:33 (primer 23) and SEQ ID NO:34 (primer 24). The kanamycin resistant marker was removed from the constructed strain using the helper plasmid pAH129-cat according to the previously reported technique (Andreeva I G et al. FEMS Microbiol Lett. 2011; 318(1):55-60, which is incorporated herein by reference in its entirety). The resulting strain SC17(0)Δcrt::attBₚₕᵢ₈₀ was verified by PCR using synthesized oligonucleotides consisting of the nucleotide sequences of SEQ ID NO:33 (primer 23) and SEQ ID NO:34 (primer 24). Maps of the resulting modified genomes ΔampC::attBₚₕᵢ₈₀, ΔampH::attBₚₕᵢ₈₀ and Δcrt::attBₚₕᵢ₈₀ are shown in FIGS. 7A, 7B and 7C, respectively.

The above plasmid pAH162-Ptac-mvk (M. paludicola) was integrated into genome of SC17(0)Δcrt::attBₚₕᵢ₈₀ according to the previously reported protocol (Andreeva I G et al. FEMS Microbiol Lett. 2011; 318(1):55-60, which is incorporated herein by reference in its entirety). The integration of the plasmid was confirmed by the polymerase chain reaction using synthesized oligonucleotides consisting of the nucleotide sequences of SEQ ID NO:31 (primer 21), SEQ ID NO:33 (primer 23). SEQ ID NO:32 (primer 22) and SEQ ID NO:34 (primer 24). As a result, a strain SC17(0)Δcrt::pAH162-P_{tac}-mvk (M. paludicola) was obtained. A map of the modified genome Δcrt::pAH162-P_{tac}-mvk (M. paludicola) is shown in FIG. 8A.

Subsequently, transfer from SC17(0)Δcrt::pAH162-P_{tac}-mvk (M. paludicola) to SC17(0) ΔampC::attBₚₕᵢ₈₀ ΔampH::attBₚₕᵢ₈₀ was carried out via the electroporation of genomic DNA (Katashkina J I et al. BMC Mol Biol. 2009; 10: 34, which is incorporated herein by reference in its entirety). The resulting strain was removed from a vector portion of the integrative plasmid pAH162-Ptac-mvk (M. paludicola) using the previously reported helper plasmid pMW-intxis-cat (Katashkina J I et al. BMC Mol Biol. 2009; 10: 34, which is incorporated herein by reference in its entirety). As a result, the marker-deleted strain SC17(0) ΔampH::attB_{φ80} ΔampC::attB_{φ80} Δcrt::P_{tac}-mvk (M. paludicola) was obtained. A map of the modified genome Δcrt::P_{tac}-mvk (M. paludicola) is shown in FIG. 8B.

(1-3-2) Construction of Strain SWITCH-Pₐᵣₐ

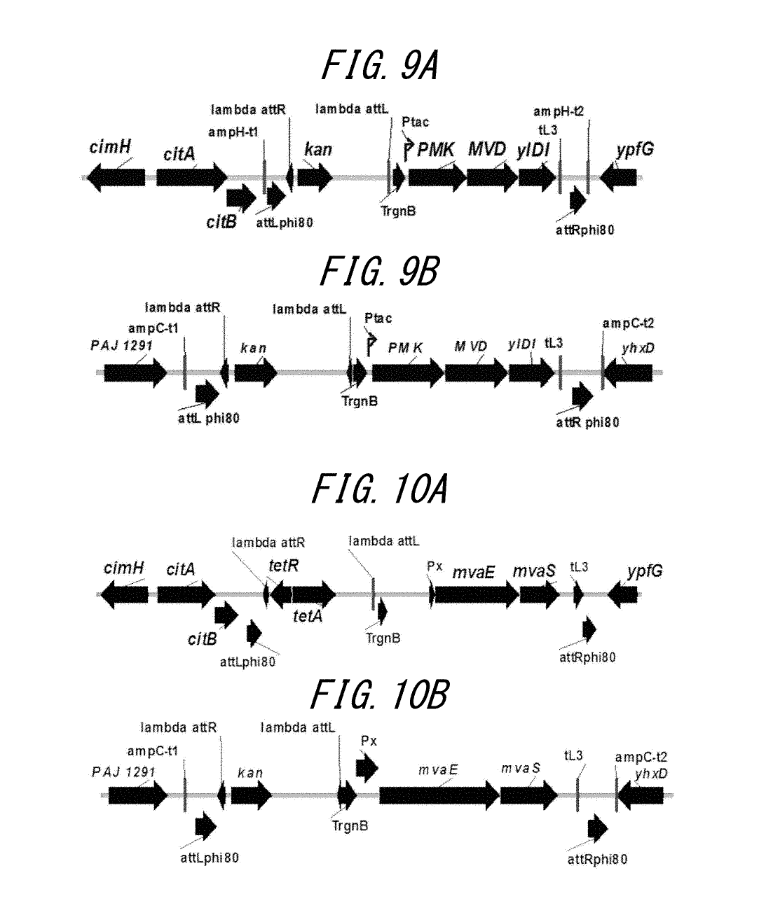

The plasmid pAH162-Km-P_{tac}-KDvI was integrated into a chromosome of the strain SC17(0)ΔampH::attB_{φ80} ΔampC::attB_{φ80} Δcrt::P_{tac}-mvk (M. paludicola)/pAH123-cat according to the previously reported protocol (Andreeva I G et al. FEMS Microbiol Lett. 2011; 318(1): 55-60, which is incorporated herein by reference in its entirety). After electrophoresis, cells were seeded on the LB agar containing 50 mg/L of kanamycin. Grown Km^{R} clones were tested by the polymerase chain reactions using synthesized oligonucleotides consisting of the nucleotide sequences of SEQ ID NO:23 (primer 13) and SEQ ID NO:27 (primer 17), and SEQ ID NO:23 (primer 13) and SEQ ID NO:29 (primer 19) as the primers. A strain holding the plasmid pAH162-Km-P_{tac}-KDyI integrated into ΔampH::attB_{φ80} or pC::attB_{φ80}m was selected. Maps of the modified genomes ΔampH::pAH162-Km-P_{tac}-KDyI and ΔampC::pAH162-Km-P_{tac}-KDyI are shown in FIGS. 9A and 9B.

pAH162-Pₐᵣₐ-mvaES was inserted into a chromosome of recipient strains SC17(0) ΔampC::pAH162-Km-P^{tac}-KDyI ΔampH::attBₚₕᵢ₈₀ Δcrt::P_{tac}-mvk (M. paludicola) and SC17(0) ΔampC::attBₚₕᵢ₈₀ ΔampH::pAH162-Km-P_{tac}-KDyI Δcrt::P_{tac}-mvk (M. paludicola) using the helper plasmid pAH123-cat according to the previously reported protocol (Andreeva I G et al. FEMS Microbiol Lett. 2011; 318(1): 55-60, which is incorporated herein by reference in its entirety). As a result, two sets of strains designated as SWITCH-Pₐᵣₐ-1 and SWITCH-Pₐᵣₐ-2 were obtained. Maps of the modified genomes ΔampH::pAH162-Pₐᵣₐ-mvaES and ΔampC::pAH162-Pₐᵣₐ-mvaES are shown in FIGS. 10A and 10B.

The plasmid of the isoprene synthase was transduced as follows.

Competent cells of SWITCH-Pₐᵣₐ-1 were adjusted according to an ordinary method, and subsequently pSTV28-P_{tac}-ispSM (US2014113344A1) was introduced thereto, and was evenly applied onto the LB plate containing 60 mg/L of chloramphenicol and cultured 37° C. for 18 hours. Subsequently, transformants that exhibited resistance to chloramphenicol were obtained from the resulting plates. The resulting transformants were designated as P. ananatis isoprene-producing bacterium and SWITCH-Pₐᵣₐ-1/pSTV28-P_{tac}-ispSM, respectively.

(1-4) Production of Isoprene Via Isoprene Fermentation

(1-4-1) Cultivation of P. ananatis Isoprene-Producing Bacterium SWITCH-Pₐᵣₐ-1/pSTV28-P_{tac}-ispSM

SWITCH-Pₐᵣₐ-1/pSTV28-P_{tac}-ispSM was used as a microbe having isoprene productivity. SWITCH-Pₐᵣₐ-1/pSTV28-P_{tac}-ispSM was applied onto an LB plate containing 60 mg/L of chloramphenicol and cultured at 34° C. for 16 hours.

0.3 L of the glucose medium as shown in the following Table 1 was placed in three 1 L volume fermenters, respectively, and microbial cells sufficiently grown on one plate were inoculated thereto to start the cultivation. A culture condition was pH 7.0 (controlled with ammonia gas), 30° C., ventilation of 150 mL/minute, and stirring such that an oxygen concentration in the medium was 5% or higher. Note that in the present example, the glucose medium as shown in Table 1 was prepared for 0.15 L of Group A and 0.15 L of Group B, and they were heated and sterilized at 115° C. for 10 minutes. After cooling, Group A and Group B were mixed, and chloramphenicol (60 mg/L) was added thereto to use as the medium.

TABLE 1

| | |
|---|---|
| Component | Concentration |

Group A

| | | |
|---|---|---|
| Glucose | 40 | g/L |
| Magnesium sulfate heptahydrate | 2.0 | g/L |

Group B

| | | |
|---|---|---|
| Ammonium sulfate | 2.0 | g/L |
| Potassium dihydrogenphosphate | 2.0 | g/L |
| Iron sulfate heptahydrate | 20 | mg/L |
| Manganese sulfate pentahydrate | 20 | mg/L |
| Yeast extracts | 4.0 | g/L |

When all of the glucose contained in the culture medium was consumed, 400 mL of the culture broth was charged into 20 L of a glucose culture medium described in Table 2 below in two fermentation tanks having a volume of 50 L, respectively. A culture condition was pH 7.0 (controlled with ammonia gas), 30° C., ventilation of 10 L/minute, and stirring such that an oxygen concentration in the medium was 5% or higher. Note that in the present example, the glucose medium as shown in Table 2 was prepared for 10 L of Group A and 10 L of Group B, and they were heated and sterilized at 115° C. for 10 minutes. After cooling, Group A and Group B were mixed, and chloramphenicol (60 mg/L) was added thereto to use as the medium. During the culture, glucose adjusted to have a concentration of 500 g/L was continuously added so that the glucose concentration of the culture medium was kept at 10 g/L or more.

TABLE 2

| | |
|---|---|
| Component | Concentration |

Group A

| | | |
|---|---|---|
| Glucose | 80 | g/L |
| Magnesium sulfate heptahydrate | 2.0 | g/L |

Group B

| | | |
|---|---|---|
| Ammonium sulfate | 2.0 | g/L |
| Potassium dihydrogenphosphate | 2.0 | g/L |
| Iron sulfate heptahydrate | 20 | mg/L |
| Manganese sulfate pentahydrate | 20 | mg/L |
| Yeast extracts | 4.0 | g/L |

The SWITCH-Pₐᵣₐ-1/pSTV28-P_{tac}-ispSM used in this example expresses the mevalonate pathway upstream gene under the arabinose inducible promoter and thus the isoprene production amount was significantly increased by the presence of L-arabinose (manufactured by Wako Pure Chemical Industries, Ltd.). In this Example, L-arabinose was added to the glucose culture medium described in Table 2 so that the final concentration of L-arabinose was 20 mM and whereby the isoprene production phase was induced.

The isoprene concentration of the fermented gas was measured with a multi gas analyzer (“F10”, manufactured by GASERA Ltd.). Isoprene was detected after 6 hours from the start of the culture. The isoprene concentration of the fermented gas reached about 800 ppm after 30 hours from the start of the culture. Thereafter, the fermented gas containing isoprene at a concentration of about 800 ppm was supplied to the isoprene recovery apparatus until 76 hours after the start of the culture. At such time, the culture was terminated.

(1-4-2) Recovery of Isoprene Contained in Fermented Gas

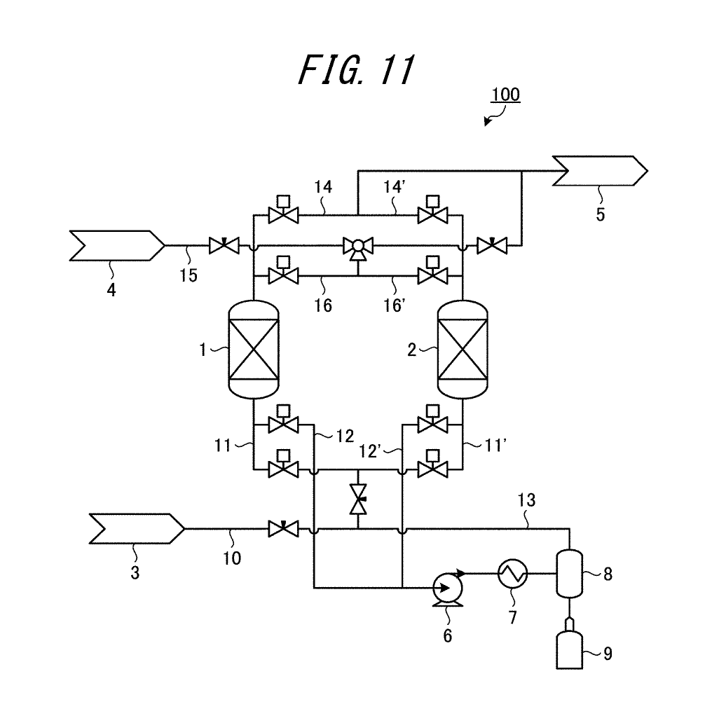

In recovery of isoprene contained in fermented gas, isoprene recovery apparatus illustrated in FIG. 11 was used. A porous adsorbent layers (not illustrated) using a hydrophobic silica gel (S-6, manufactured by Fuji Silysia Chemical Ltd.) was provided in each of a first adsorption tower 1 and a second adsorption tower 2. The porous adsorbent layers were provided in a pre-coated state by blowing with gas having an isoprene concentration of 1.0% by volume.

In the adsorption step, the fermented gas generated from the fermentation tank was used as the feed gas 3. After the feed gas 3 was subjected to dehydration treatment and organic substance removal treatment, the treated feed gas 3 was supplied to the first adsorption tower 1 (or the second adsorption tower 2) through the feed gas supply pipes 10 and 11 (or 10 and 11′). Conditions at the time of adsorption were a temperature of 25° C., a pressure of 101.3 kPa, and a feed gas linear velocity of 1.8 cm/sec. The isoprene concentration of the discharged gas 5 emitted to the atmosphere after the adsorption step was measured with a multi-gas analyzer (F10, manufactured by GASERA).

In a desorption step, nitrogen gas was used as the purge gas 4, and the purge gas 4 was supplied to the first adsorption tower 1 (or the second adsorption tower 2) through purge gas supply pipes 15 and 16 (or 15 and 16′). At the time of desorption, the pressure in the adsorption towers 1, 2 was reduced to 3.3 kPa with the vacuum pump 6. Moreover, the linear velocity of purge gas was 1.6 cm/sec. Cooling water having a temperature of 10° C. was circulated in a condenser 7 to cool the gas containing isoprene. Thereafter, the cooled gas was separated with a separator 8 to recover the liquid isoprene 9.

Note that the isoprene recovery apparatus 100 may, as illustrated in FIG. 11, be provided with a return pipe 13 and discharge pipes 14, 14′.

The isoprene recovery apparatus 100 was operated out by alternately switching the adsorption step and the desorption step (the number of iterations of each step: 300 times; switching time of each step: 15 minutes).

Via the operations above, 111 g in total of isoprene was recovered. The recovered isoprene (isoprene-containing composition) was used in study of polymerization reaction.

(2) Purification of Isoprene-Containing Composition

The isoprene prepared with the aforementioned fermentation method was purified with column chromatography. As a column filler (solid), used was an activated alumina (alumina oxide (active) (granular), manufactured by Kanto Chemical Co., Inc.), and molecular sieves (molecular sieves 4A, manufactured by Kanto Chemical Co., Inc.), and as an elute (liquid), used was hexane.

Details of the purification condition were as shown in Table 3 (described below).

(3) Analysis of Isoprene-Containing Composition

In order to certify the quality of the isoprene provided to the following manufacture of polyisoprene, the isoprene prepared with the aforementioned fermentation method was analyzed with gas chromatography.

The samples were analyzed by using a capillary column for gas chromatography (HP-5MSUI (length: 30 m, internal diameter: 0.25 mm, film thickness: 1 μm), manufactured by Agilent Technologies, Inc.) and a gas chromatography apparatus equipped with a hydrogen flame ionization detector (FID) (7820A, manufactured by Agilent Technologies, Inc.) under a condition of: holding at a column temperature of 35° C. for 3 minutes, then heating to 300° C. at 25° C./min, and then holding for 13 minutes at 300° C.; pressure: 19 kPa; column flow: 3 mL/min; influx method: split 100:1; injection volume of sample into column: 0.1 μL; inlet temperature: 250° C.; detector temperature: 230° C.

In the present example, in particular, ethyl acetate and 3-methylfuran was analyzed as main components of the impurities. In the case where both ethyl acetate and 3-methylfuran were detected, the total amount thereof was calculated, and in the case where either one was detected, the amount of each was calculated respectively (see Table 3).

A commercially available isoprene (proportion: 0.681) was diluted 10, 100, 1000, 10000 and 100000 times with cooled methanol, and adjusted with a isoprene solution for standard sample. Subsequently, 1 μL of each solution for standard sample was respectively added into vials added with 1 mL of water, to thereby obtain isoprene standard samples.

(4) Manufacture of Polyisoprene

3.5 g of purified isoprene was added into a sufficiently dried 2 L stainless reactor.

On the other hand, within a globe box under a nitrogen atmosphere, in a glass container, 40.0 μmol of tris bistrimethylsilylamide gadolinium (Gd[N(SiMe₃)₂]₃) (component (A)), 200 μmol of tributyl aluminum (component (B)), 40.0 μmol of bis(2-diphenylphosphinophenyl) amine (BDPA), 5 g of toluene, 40.0 μmol of triphenylcarbonium tetrakis(pentafluorophenyl) borate (Ph₃C⁺B⁻(C₆F₅)₄) (component (E)) and 35 g of n-hexane were added as the polymerization catalyst composition.

Then, the polymerization catalyst composition was removed from the glove box, and was added into the 2 L reactor containing isoprene. The reaction system was maintained at 50° C. for 120 minutes to perform polymerization reaction of isoprene. Subsequently, by adding a large amount of methanol into the reactor, the polymerization reaction was terminated, the reaction product was precipitated and separated, and further vacuum-dried at 50° C. to obtain a polymer A (yield: 3.3 g).

By using a gel permeation chromatography (GPC) (GPC apparatus: HLC-8220GPC manufactured by Tosoh Corporation, column: 2 TSKgel GMH_{XL} manufactured by Tosoh Corporation, detector: a differential refractometer (RI)), on the basis of monodisperse polystyrene, the number average molecular weight (Mn) and the molecular weight distribution (Mw/Mn) of polystyrene equivalent of the manufactured polyisoprene were calculated. Note that the measurement temperature was 40° C., and THF was used as an elution solvent.

A rubber composition containing the manufactured polyisoprene was prepared, and by vulcanizing the rubber composition, a vulcanized rubber was obtained. Then, a sample of this vulcanized rubber was subjected to tensile test according to JIS K 6251, and to measure its tensile breaking strength (TB) at room temperature. The results thereof are shown as being indexed with a score of 100 representing Comparative Example 1. The result was as shown in Table 3. An index serving as a comparative evaluation was calculated with a score of 100 representing a conventional example. The evaluation results were as shown in Table 3. A higher index value indicates a higher tensile breaking strength (TB).

TABLE 3

| | | | | Comparative | Comparative | Comparative | Comparative | |
|---|---|---|---|---|---|---|---|---|
| | | | | Example 1 | Example 2 | Example 3 | Example 4 | Example 1 |
| Specs of | Isoprene- | Type of isoprene | | Synthesized | Synthesized | Fermented | Fermented | Fermented |
| method | containing | Degree of isoprene purification | | Highly purified | Highly purified | Highly purified | Roughly | Roughly |
| for | composition | | | | | | purified | purified |
| producing | | Condition of | Activated alumina | — | — | 15 | 5 | 5 |
| polyisoprene | | isoprene | amount (g) | | | | | |
| | | purification | Silica gel amount | — | — | 15 | 5 | 5 |
| | | | (g) | | | | | |
| | | | Purification time | — | — | 1 | 1 | 1 |
| | | | (time) | | | | | |
| | | | Purification | — | — | RT | RT | RT |
| | | | temperature | | | | | |
| | | | (° C.) | | | | | |
| | | Isoprene | Isoprene (mass %) | 100 | 100 | 99.97 | 99.75 | 99.75 |
| | | Oxygen-containing | Ethyl acetate and | 0 | 0 | 0.03 | 0.25 | 0.25 |
| | | neutral compound | 3-methylfuran | | | | | |
| | | | (total) (mass %) | | | | | |
| | | | Ethyl acetate | 0 | 0 | — | — | — |
| | | | (mass %) | | | | | |
| | | | 3-methylfuran | 0 | 0 | — | — | — |
| | | | (mass %) | | | | | |
| | Total (mass %) | | | 100 | 100 | 100 | 100 | 100 |
| | Polymerization | Rare earth element | Gd[N(SiMe₃)₂]₃ | 40 | 40 | 40 | 40 | 40 |
| | catalyst | compound | (μmol) | | | | | |
| | composition | Component (A) | Versatic acid Nd | 0 | 0 | 0 | 0 | 0 |
| | | | (μmol) | | | | | |
| | | Organometallic | Tr¹BuAl (μmol) | 100 | 200 | 100 | 100 | 200 |
| | | compound | Tr¹BuAl (g) | 0.02 | 0.04 | 0.02 | 0.02 | 0.04 |
| | | Component (B) | Isoprene-containing | 3.5 | 3.5 | 3.5 | 3.5 | 3.5 |
| | | | composition (g) | | | | | |
| | | | Ratio of TriBuAl to | 0.57 | 1.13 | 0.57 | 0.57 | 1.13 |
| | | | isoprene-containing | | | | | |
| | | | composition | | | | | |
| | | | (parts by mass) | | | | | |
| | | Ionic compound | Ph₂CB(C₆F₅)₄ | 40 | 40 | 40 | 40 | 40 |
| | | Component (E) | (μmol) | | | | | |
| | | Compound capable | BDPA (μmol) | 40 | 40 | 40 | 40 | 40 |
| | | of serving as an | | | | | | |
| | | anionic ligand | | | | | | |
| | | Component (F) | | | | | | |
| | | Solvent | Toluene (g) | 5 | 5 | 5 | 5 | 5 |
| | | | Hexene (g) | 35 | 35 | 35 | 35 | 35 |

| | | | | | |
|---|---|---|---|---|---|
| Capability of polymerization | Capable | Capable | Capable | Incapable | Capable |

| | | | | | | |
|---|---|---|---|---|---|---|
| Specs of polyisoprene | Mn (×10³) | 1220 | 1150 | 1190 | — | 1640 |
| | Mw (×10³) | 2367 | 2220 | 2309 | — | 3150 |
| | Mw/Mn | 1.94 | 1.93 | 1.94 | — | 1.92 |
| | TB | 100 | 99 | 100 | — | 110 |

| | | | | Example 2 | Example 3 | Example 4 | Example 5 | Example 6 | Example 7 |
|---|---|---|---|---|---|---|---|---|---|
| Specs of | Isoprene- | Type of isoprene | | Fermented | Fermented | Fermented | Fermented | Fermented | Synthesized |
| method | containing | Degree of isoprene purification | | Roughly | Roughly | Roughly | Roughly | Roughly | Highly purified |
| for | composition | | | purified | purified | purified | purified | purified | |
| producing | | Condition of | Activated alumina | 15 | 5 | 5 | 5 | 5 | — |
| polyisoprene | | isoprene | amount (g) | | | | | | |
| | | purification | Silica gel amount | 15 | 5 | 5 | 5 | 5 | — |
| | | | (g) | | | | | | |
| | | | Purification time | 0.5 | 0.5 | 0.35 | 1 | 1 | — |
| | | | (time) | | | | | | |
| | | | Purification | RT | RT | RT | RT | RT | — |
| | | | temperature | | | | | | |
| | | | (° C.) | | | | | | |
| | | Isoprene | Isoprene (mass %) | 99.95 | 99.0 | 98.4 | 99.75 | 99.75 | 99.75 |
| | | Oxygen-containing | Ethyl acetate and | 0.05 | 1.0 | 1.6 | 0.25 | 0.25 | — |
| | | neutral compound | 3-methylfuran | | | | | | |
| | | | (total) (mass %) | | | | | | |
| | | | Ethyl acetate | — | — | — | — | — | 0.25 |
| | | | (mass %) | | | | | | |
| | | | 3-methylfuran | — | — | — | — | — | 0 |
| | | | (mass %) | | | | | | |
| | Total (mass %) | | | 100 | 100 | 100 | 100 | 100 | 100 |
| | Polymerization | Rare earth element | Gd[N(SiMe₃)₂]₃ | 40 | 40 | 40 | 40 | 40 | 40 |
| | catalyst | compound | (μmol) | | | | | | |
| | composition | Component (A) | Versatic acid Nd | 0 | 0 | 0 | 0 | 0 | 0 |
| | | | (μmol) | | | | | | |
| | | Organometallic | Tr¹BuAl (μmol) | 200 | 200 | 300 | 150 | 300 | 200 |
| | | compound | Tr¹BuAl (g) | 0.04 | 0.04 | 0.06 | 0.03 | 0.06 | 0.04 |
| | | Component (B) | Isoprene-containing | 3.5 | 3.5 | 3.5 | 3.5 | 3.5 | 3.5 |
| | | | composition (g) | | | | | | |
| | | | Ratio of TriBuAl to | 1.13 | 1.13 | 1.70 | 0.85 | 1.70 | 1.13 |
| | | | isoprene-containing | | | | | | |
| | | | composition | | | | | | |
| | | | (parts by mass) | | | | | | |
| | | Ionic compound | Ph₂CB(C₆F₅)₄ | 40 | 40 | 40 | 40 | 40 | 40 |
| | | Component (E) | (μmol) | | | | | | |
| | | Compound capable | BDPA (μmol) | 40 | 40 | 40 | 40 | 40 | 40 |
| | | of serving as an | | | | | | | |
| | | anionic ligand | | | | | | | |
| | | Component (F) | | | | | | | |
| | | Solvent | Toluene (g) | 5 | 5 | 5 | 5 | 5 | 5 |
| | | | Hexene (g) | 35 | 35 | 35 | 35 | 35 | 35 |

| | | | | | | |
|---|---|---|---|---|---|---|
| Capability of polymerization | Capable | Capable | Capable | Capable | Capable | Capable |

| | | | | | | | |
|---|---|---|---|---|---|---|---|
| Specs of polyisoprene | Mn (×10³) | 1250 | 1300 | 1250 | 1320 | 1360 | 1620 |
| | Mw (×10³) | 2450 | 2522 | 2058 | 2530 | 2665 | 3159 |
| | Mw/Mn | 1.96 | 1.94 | 1.65 | 1.92 | 1.96 | 1.95 |
| | TB | 100 | 102 | 100 | 105 | 105 | 107 |

| | | | | Example 8 | Example 9 | Example 10 | Example 11 |
|---|---|---|---|---|---|---|---|
| Specs of | Isoprene- | Type of isoprene | | Synthesized | Fermented | Fermented | Fermented |
| method | containing | Degree of isoprene purification | | Highly purified | Roughly | Roughly | Roughly |
| for | composition | | | | purified | purified | purified |
| producing | | Condition of | Activated alumina | — | 15 | 5 | 5 |
| polyisoprene | | isoprene | amount (g) | | | | |
| | | purification | Silica gel amount | — | 15 | 5 | 5 |
| | | | (g) | | | | |
| | | | Purification time | — | 0.5 | 0.5 | 1 |
| | | | (time) | | | | |
| | | | Purification | — | RT | RT | RT |
| | | | temperature | | | | |
| | | | (° C.) | | | | |
| | | Isoprene | Isoprene (mass %) | 99.75 | 99.95 | 99.0 | 99.75 |
| | | Oxygen-containing | Ethyl acetate and | — | 0.05 | 1.0 | 0.25 |
| | | neutral compound | 3-methylfuran | | | | |
| | | | (total) (mass %) | | | | |
| | | | Ethyl acetate | 0 | — | — | — |
| | | | (mass %) | | | | |
| | | | 3-methylfuran | 0.25 | — | — | — |
| | | | (mass %) | | | | |

| Total (mass %) | | | 100 | 100 | 100 | 100 |
|---|---|---|---|---|---|---|
| Polymerization | Rare earth element | Gd[N(SiMe₃)₂]₃ | 40 | 40 | 40 | 0 |
| catalyst | compound | (μmol) | | | | |
| composition | Component (A) | Versatic acid Nd | 0 | 0 | 0 | 40 |
| | | (μmol) | | | | |
| | Organometallic | Tr¹BuAl (μmol) | 200 | 150 | 300 | 200 |
| | compound | Tr¹BuAl (g) | 0.04 | 0.03 | 0.06 | 0.04 |
| | Component (B) | Isoprene-containing | 3.5 | 3.5 | 3.5 | 3.5 |
| | | composition (g) | | | | |
| | | Ratio of TriBuAl to | 1.13 | 0.85 | 1.70 | 1.13 |
| | | isoprene-containing | | | | |
| | | composition | | | | |
| | | (parts by mass) | | | | |
| | Ionic compound | Ph₂CB(C₆F₅)₄ | 40 | 40 | 40 | 40 |
| | Component (E) | (μmol) | | | | |
| | Compound capable | BDPA (μmol) | 40 | 40 | 40 | 40 |
| | of serving as an | | | | | |
| | anionic ligand | | | | | |
| | Component (F) | | | | | |
| | Solvent | Toluene (g) | 5 | 5 | 5 | 5 |
| | | Hexene (g) | 35 | 35 | 35 | 35 |

| | | | | |
|---|---|---|---|---|
| Capability of polymerization | Capable | Capable | Capable | Capable |

| | | | | | |
|---|---|---|---|---|---|
| Specs of polyisoprene | Mn (×10³) | 1590 | 1553 | 1515 | 1580 |
| | Mw (×10³) | 3159 | 3028 | 3015 | 3239 |
| | Mw/Mn | 1.99 | 1.95 | 1.99 | 2.05 |
| | TB | 107 | 107 | 106 | 110 |

### Examples 2 to 11, Comparative Examples 1 to 4

The manufacture of polyisoprene was performed the same as Example 1 except for the points of condition as shown in Table 3.

Comparison of Examples 1 to 11 and Comparative Examples 1 to 4 showed that if the compounding amount of the organometallic compound was 0.6 to 3.0 parts by mass per 100 parts by mass of the isoprene-containing composition, a polyisoprene having a comparatively high molecular weight may be manufactured, and thereby, it was possible to obtain the desired effect of this disclosure, such that the strength of the manufactured synthesized polyisoprene may be enhanced. Here, the comparison between Examples 1 to 3, 5 to 11 and Example 4 showed that if the compounding amount of the oxygen-containing neutral compound was 0.05 to 1.0 parts by mass per 100 parts by mass of the isoprene-containing composition, the aforementioned effect of this disclosure was particularly significant.

Moreover, the comparison of the comparison result of Examples 1 to 6, 9 to 11 and Comparative Example 4 and the comparison result of Examples 7, 8 and Comparative Examples 1, 2 showed that the aforementioned effect of this disclosure may be obtained beneficially particularly in the case of using a fermented isoprene in which an oxygen-containing neutral compound remains after the purification.

### INDUSTRIAL APPLICABILITY

According to the method for producing polyisoprene of this disclosure, it is possible to produce a polyisoprene having a comparatively high molecular weight, and to thereby improve the produced synthesized polyisoprene.

### REFERENCE SIGNS LIST

1 first adsorption tower
2 second adsorption tower
3 fermented gas containing isoprene (feed gas)
4 purge gas
5 discharged gas
6 vacuum pump
7 condenser
8 separator
9 recovered isoprene (liquid isoprene)
10,11,11′ feed gas supply pipe
12,12′ isoprene-containing purge gas supply pipe
13 return pipe
14,14′ discharge pipe
15,16,16′ purge gas supply pipe
100 isoprene recovery apparatus

### [Indication of Microbe Deposit]

Biologicals depository: VKPM Russian National Collection of Industrial Microorganisms

Addressing: FGUP GosNII Genetika, 1 Dorozhny proezd, 1, Moscow 117545, Russian Federation

Deposit number: VKPM B-9246

Deposit date: Sep. 21, 2005

1. A method for producing polyisoprene comprising: polymerizing isoprene by using an isoprene-containing composition containing isoprene and an oxygen-containing neutral compound, and a polymerization catalyst composition containing a rare earth element compound and an organometallic compound, wherein:
a compounding amount of the organometallic compound is 0.6 to 3.0 parts by mass per 100 parts by mass of the isoprene-containing composition.

2. The method for producing polyisoprene according to claim 1, wherein:
a compounding amount of the oxygen-containing neutral compound is 0.05 to 1.0 parts by mass per 100 parts by mass of the isoprene-containing composition.

3. The method for producing polyisoprene according to claim 1, wherein:
the isoprene-containing composition is prepared via a fermentation method.

4. The method for producing polyisoprene according to claim 1, wherein:
the oxygen-containing neutral compound is selected from the group consisting of an ether, an ester or a ketone.

5. The method for producing polyisoprene according to claim 1, wherein:
a number-average molecular weight Mn of the polyisoprene is 1,000,000 or more.

6. The method for producing polyisoprene according to claim 1, wherein:
the number-average molecular weight Mn of the polyisoprene is 1,300,000 or more.

7. The method for producing polyisoprene according to claim 1, wherein:
a molecular weight distribution Mw/Mn of the polyisoprene is 2.5 or less.

8. The method for producing polyisoprene according to claim 1, wherein:
a cis-1,4-bond content of the polyisoprene is 98% or more.

9. The method for producing polyisoprene according to claim 1, wherein:
the rare earth element compound is a rare earth element-containing compound or a reaction product of the rare earth element-containing compound and a Lewis base, the rare earth element-containing compound or reaction product having metal-nitrogen bond (M-N bond), and
the rare earth element-containing compound is a compound containing scandium, yttrium or a lanthanoid element selected from elements of atomic numbers 57 to 71.

10. The method for producing polyisoprene according to claim 1, wherein:
the organometallic compound is a compound represented with
the following formula (S2)
YR⁴ₐR⁵_{b}R⁶_{c} (S2)
where Y is a metallic element selected from the group consisting of Group 1, Group 2, Group 12 and Group 13; R⁴ and R⁵ are C1 to C10 hydrocarbon group or hydrogen atom, and may be either identical or different; R⁶ is a C1 to C10 hydrocarbon group; R⁶ may be either identical to or different from the R⁴ or R⁵; if Y is a Group 1 metallic element, a is 1, and b, c are 0; if Y is a Group 2 metallic element or a Group 12 metallic element, a and b are 1, and c is 0; and if Y is a Group 13 metallic element, a, b and c are 1; or
the following formula (S3)
AlR⁷R⁸R⁹ (S3)
where R⁷ and R⁸ are C1 to C10 hydrocarbon group or hydrogen atom, and may be either identical or different; R⁹ is a C1 to C10 hydrocarbon group; and R⁹ may be either identical to or different from the R⁷ or R⁸.

11. The method for producing polyisoprene according to claim 1, wherein:
the polymerization catalyst composition is at least one compound selected from the group consisting of an aluminoxane compound, a halogen compound, an ionic compound, or a compound capable of serving as an anionic ligand.

12. The method for producing polyisoprene according to claim 2, wherein:
the isoprene-containing composition is prepared via a fermentation method.

13. The method for producing polyisoprene according to claim 2, wherein:
the oxygen-containing neutral compound is selected from the group consisting of an ether, an ester or a ketone.

14. The method for producing polyisoprene according to claim 2, wherein:
a number-average molecular weight Mn of the polyisoprene is 1,000,000 or more.

15. The method for producing polyisoprene according to claim 2, wherein:
the number-average molecular weight Mn of the polyisoprene is 1,300,000 or more.

16. The method for producing polyisoprene according to claim 2, wherein:
a molecular weight distribution Mw/Mn of the polyisoprene is 2.5 or less.

17. The method for producing polyisoprene according to claim 2, wherein:
a cis-1,4-bond content of the polyisoprene is 98% or more.

18. The method for producing polyisoprene according to claim 2, wherein:
the rare earth element compound is a rare earth element-containing compound or a reaction product of the rare earth element-containing compound and a Lewis base, the rare earth element-containing compound or reaction product having metal-nitrogen bond (M-N bond), and
the rare earth element-containing compound is a compound containing scandium, yttrium or a lanthanoid element selected from elements of atomic numbers 57 to 71.

19. The method for producing polyisoprene according to claim 2, wherein:
the organometallic compound is a compound represented with
the following formula (S2)
YR⁴ₐR⁵_{b}R⁶_{c} (S2)
where Y is a metallic element selected from the group consisting of Group 1, Group 2, Group 12 and Group 13; R⁴ and R⁵ are C1 to C10 hydrocarbon group or hydrogen atom, and may be either identical or different; R⁶ is a C1 to C10 hydrocarbon group; R⁶ may be either identical to or different from the R⁴ or R⁵; if Y is a Group 1 metallic element, a is 1, and b, c are 0; if Y is a Group 2 metallic element or a Group 12 metallic element, a and b are 1, and c is 0; and if Y is a Group 13 metallic element, a, b and c are 1; or
the following formula (S3)
AlR⁷R⁸R⁹ (S3)
where R⁷ and R⁸ are C1 to C10 hydrocarbon group or hydrogen atom, and may be either identical or different; R⁹ is a C1 to C10 hydrocarbon group; and R⁹ may be either identical to or different from the R⁷ or R⁸.

20. The method for producing polyisoprene according to claim 2, wherein:
the polymerization catalyst composition is at least one compound selected from the group consisting of an aluminoxane compound, a halogen compound, an ionic compound, or a compound capable of serving as an anionic ligand.
